(19) United States
(12) Patent Application Publication
(10) Pub. No.: US 2004/0086911 A1
(43) Pub. Date: May 6, 2004

(54) Inhibition of gene expression in vertebrates using double-stranded RNA (RNAi)

(75) Inventors: Olga A. Cabello, Houston, TX (US); Paul A. Overbeek, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX

(21) Appl. No.: 10/602,235

(22) Filed: Jun. 24, 2003

Related U.S. Application Data
(60) Provisional application No. 60/390,972, filed on Jun. 24, 2002.

(51) Int. Cl. C12Q 1/68; C07H 21/02; A61K 48/00

(52) U.S. Cl. 435/6; 514/44; 536/23.1

(57) ABSTRACT

The present invention is directed to RNA interference using novel compositions and methods. In particular embodiments, the RNA compositions comprise double strand regions interrupted with non-complementary regions, wherein the RNA compositions are effective for regulation of transcription. In specific embodiments, transcription of a target nucleic acid sequence to which the RNA composition is directed is reduced or inhibited, such as by inducing destruction of at least one transcript. In other embodiments, multiple target nucleic acid sequences are targeted by the RNA compositions of the present invention.

### CROSS-REFERENCE TO RELATED APPLICATIONS

The present invention claims priority to U.S. Provisional Patent Application No. 60/390,972, filed Jun. 24, 2002, which is incorporated by reference herein in its entirety.

### STATEMENT REGARDING FEDERALLY SPONSORED RESEARCH OR DEVELOPMENT

Federal funds pursuant to grant numbers R03EY14271, KO1HL03850, RO1EY10448, PO1HL49953, RO1AR45316 and RO3AG18809 were utilized in the present invention. The United States Government may have certain rights in the invention.

### FIELD OF THE INVENTION

The present invention is directed to the fields of molecular biology and cell biology, and particularly gene expression. Specifically, the present invention regards reduction or inhibition of transcription. More specifically, the reduction or inhibition of transcription utilizes a double stranded RNA molecule comprising at least one region of non complementarity.

### BACKGROUND OF THE INVENTION

Targeted inactivation of mouse genes is currently accomplished by homologous recombination in ES cells. This protocol requires the creation and characterization of embryonic stem (ES) cell clones, the transfer of ES cells to recipient blastocysts to generate germline chimeras, and at least two generations of matings to produce null animals. Tissue specific knock-outs require additional genetic engineering in ES cells followed by extra matings to families expressing Cre or FLP recombinases. In all cases, gene inactivation is irreversible once induced in the live mouse. Clearly, it would be useful to have a strategy for both dominant and reversible tissue-specific gene silencing.

The term RNA interference (RNAi) refers to the suppression of gene expression by introduction of double stranded RNA (dsRNA) molecules homologous to a target gene (Fire et al., 1998; Carthew, 2001; Zamore, 2001; Hammond et al., 2001; Sharp, 2001). RNAi was discovered by Fire and coworkers in 1998 (Fire et al., 1998) when they noted that inhibition of gene expression with antisense RNAs was much more effective when animals were injected with both sense and antisense RNAs for a targeted gene.

Although RNAi is now widely used as an experimental tool in C. elegans, not all steps in the pathway have been characterized. The initial step is the cleavage of dsRNA precursors to yield short interfering RNAs (siRNA) that are 21-22 bp in length (Elbashir et al., 2001). Cleavage involves the RNAse III-like enzyme Dicer, recently identified in the laboratory of Gregory Hannon (Bernstein et al., 2001; Hutvagner et al., 2001; Knight and Bass, 2001; Elbashir et al., 2001; Grishok et al., 2001). The siRNAs are thought to direct an RNA-degradation complex termed RISC (RNA-induced silencing complex) to the complementary endogenous transcript (Hammond et al., 2000, Zamore, 2001). Components of RISC include the RecQ DNA helicase homologue gde-3, the dsRNA-binding protein rde-4, the PAZ-Piwi domain proteins rde-1 and ago-2, the putative RNA helicases mut-6 and sde3, and the RNAseD homologue mut-7 (Zamore, personal communication and reviewed in Sharp, 2001). All these proteins exhibit functions that are compatible with a role in RNA processing. It appears that siRNAs associate with RISC as duplexes, but there is evidence that target mRNA degradation is mediated by specific hybridization with the siRNA antisense sequence (Zamore, 2001; Sharp, 2001).

In Drosophila (Hutvagner et al., 2001) and C. elegans (Grishok et al., 2001) endogenous gene expression can be regulated by short temporal RNAs (stRNAs). These transcripts adopt a complex secondary structure containing dsRNA regions, and are cleaved to generate 21-22 base pair dsRNA intermediates (Pasquinelli et al., 2001). These intermediates are subsequently unwound in vivo. The antisense strands then hybridize with target mRNA (Moss, 2001), resulting in gene silencing by interference with translation. Two genes known to mediate their function in this fashion are let-4 and let-7 which repress let-14 and let-41 translation, respectively. (Pasquinelli, 2000, Hutvagner et al., 2001; Grishok et al., 2001). Three new reports indicate that multiple stRNA called miRNA (micro RNAs) have been identified in Drosophila, C. elegans and human cells. These findings suggest that the endogenous regulation of gene expression by short RNAs is a much more widely used than previously suspected and reinforces the idea that intermediary pathways are conserved (Lee et al., 2001; Lau et al., 2001; Lagos-quintana et al., 2001)

Several reports strongly suggest, that the basic molecular components of the RNAi pathways are conserved in mammalian cells. Svoboda et al., (2000) and Wianny et al., (2000) demonstrated that dsRNAs microinjected into fertilized mouse embryos can completely suppress expression of individual target genes, including mos, E-cadherin, and a GFP-encoding transgene. Recently, Yang et al, (2001) have shown that undifferentiated mouse ES cells have a sequence-specific RNAi activity that disappears as the cells differentiate and that cytoplasmic extracts from mammalian cells can produce short dsRNA (21-22 base pairs) from long precursors. Similar results were observed in mouse oocytes (Svoboda et al., 2001) transfected with plasmid vectors encoding inverted repeat sequences corresponding to EGFP. The RNAi pathway also appears to function in mammalian cells in culture. Reporter gene expression was shown to be suppressed in cells transfected with 21-nucleotide siRNAs (Elbashir et al., 2001). Interestingly, dsRNAs longer than 30 base pairs induced non-specific reductions in gene expression, indicating the activation of an interferon response.

A major impediment to the implementation of RNAi protocols in mice is the existence of an endogenous system that responds to dsRNA. The presence of dsRNA in mammalian cells triggers an interferon response that results in non-specific suppression of gene expression and may culminate in apoptosis (Sen, 2001; Taniguchi and Takaoka, 2001). dsRNA is not only a potent inducer of interferons and interferon-stimulated genes (ISG), but also activates PKR (protein kinase stimulated by dsRNA) (Barber, 2001; Gil and Esteban, 2000; Williams et al., 1999; Yokoyama et a., 1990). PKR phosphorylates and inactivates eIF2α (eukaryotic initiation factor 2α), thereby blocking protein synthesis.

An RNAi-based gene silencing strategy would have significant advantages over the current protocols for gene inactivation in mammals, such as mice. It would be simpler, faster, more flexible and potentially reversible. Genomic locus inactivation by mutagenesis or by genomic engineering is not readily reversible and usually produces recessive phenotypes. The strategy of the present invention is designed to produce dominant inhibition of target gene function.

Caplen et al. (2001) describes short interfering RNAs are capable of inducing gene-specific inhibition of expression in cell lines from humans and mice. The siRNAs characteristically comprised a 5 phosphate, 3′ hydroxyl, and 2 base 3′ overhangs on each strand, and they varied in size from 21-27 nucleotides in length.

Hamada et al. (2002) relates to RNAi in cultured mammalian cells, wherein a mismatch in the double stranded region of antisense strand of the siRNAs rendered a reduction in inhibition of expression.

Hasuwa et al. (2002) describe siRNA and effective gene silencing in transgenic mice and rats, wherein the siRNA species comprises 21 nucleotide sequences and a 9 nucleotide spacer sequence providing a loop structure.

Stewart et al. (2003) is directed to delivery in a lentivirus vector of cassettes expressing effective hairpin RNA targeting sequences.

Franch et al. (1999) regards antisense RNA regulation in prokaryotes, wherein the RNA/RNA interaction is facilitated by a general U-turn loop structure. Particularly, it regards a stem loop structure having a U-turn motif.

Wilson et al. (1997) demonstrates stem loop structures wherein within the stems there also comprises loops, and wherein the compositions for translational enhancement of repA expression.

WO 03/012052 is directed to compositions and methods for inhibition of expression of a target gene concerning small double stranded RNAs, particularly comprising about 15-40 nucleotides in length and having a 3′ or 5′ overhang having a length of 0-5 nucleotides on each strand, and the double strand region is substantially identical to a portion of a mRNA or transcript of a target gene.

WO 03/006477 describes nucleic acid molecules that encode RNA precursors having a first stem portion of at least 18 nucleotides that is complementary to an mRNA, a second stem portion that hybridizes to the first stem portion to form a duplex stem, and a loop portion there between.

Leirdal and Sioud, 2002 describe suppression of gene expression utilizing two self-complementary siRNAs interrupted by a single-stranded loop region.

Paul et al,. 2002 relates to siRNA having a 19-base pair siRNA stem with the two strands joined by a tightly structure loop and a U₁₋₄3′ overhang at the end of the antisense strand.

Kennerdell and Carthew, 2000 is directed to an extended hairpin-loop RNA for target gene expression regulation in Drosophila.

WO 01/68836, Ser. Nos. US/2003/0084471 and US/2002/0162126 describe methods for attenuating gene expression in a cell using dsRNA, particularly by activating Dicer or Argonaut in the cell.

WO 01/70949 is directed to reduction of translation of a target endogenous nucleotide sequence or an altered capacity for translation thereof using a genetic construct administered to a cell comprising the sequence, wherein the construct preferably comprises a sequence substantially identical to the target endogenous sequence and a sequence complementary thereto, wherein they are separated by an intron having a particular sequence.

WO 99/49029 regards modification of gene expression by administering multiple copies of a nucleic acid sequence that is substantially identical to a target gene, preferably modifying translation of a product encoded by the nucleic acid sequence. In some embodiments, the nucleic acid sequence is modified by a synthetic gene comprising multiple structural gene sequences, wherein each structural gene sequence is substantially identical to the target gene.

U.S. Pat. No. 6,573,099 is directed to genetic constructs for delaying, repressing, or reducing expression of a target gene, wherein the construct comprises at least two copies of a structural gene sequence that is substantially identical to at least a region of the target gene, wherein the structural gene sequence is regulated in a variety of specific embodiments.

WO 01/75164 and Ser. No. US/2002/0086356 concern RNA mediators of RNA interference being about 21-23 nucleotides in length and corresponding to a particular target gene, and particularly using the mediators with Drosophila embryo extract in methods of mediating mRNA degradation.

Ser. No. US/2002/0173478 regards disruption of expression related to post-transcriptional gene silencing for human cells and cell lines using dsRNA compositions.

WO 02/44321 is related to short interfering RNAs generated from long dsRNAs, wherein the short interfering RNAs are double stranded and are from 19-25 nucleotides in length and particularly have 3′ overhangs of from 1-3 nucleotides.

### BRIEF SUMMARY OF THE INVENTION

In a specific embodiment, the present invention regards inducible and reversible tissue-specific gene silencing in a mammal, such as the mouse. In one particular embodiment, the present invention regards the utilization of RNAi in transgenic mice without inducing interferons. In other specific embodiments the present invention is directed to utilization of RNAi for clinical therapy in a human.

RNA interference (RNAi)- based strategy for gene inactivation in a mammal, such as transgenic mice. RNAi is a strategy that takes advantage of a conserved endogenous pathway for gene silencing. This pathway appears to serve a dual function: to guard cells against molecular parasites, and to regulate the chronology of development. RNAi is triggered by double-stranded RNAs (dsRNA), corresponding to sense and antisense sequences of target genes. The dsRNA precursors are cleaved by Dicer RNAse into short interfering RNAs (siRNAs) that guide an RNA Induced Silencing Complex (RISC) to the target transcript. In a related mechanism, Dicer also cleaves dsRNA precursors to generate short temporal RNAs (stRNAs) that act by interfering with translation. Thus, at least two RNA-based mechanisms exist for regulation of gene expression: transcription-based regulation and translation-based regulation.

RNAi is the most powerful technique available for the functional analysis of C.elegans and Drosophila genomes, and systematic targeted inactivation projects are under way. The establishment of RNAi-based gene inactivation technologies in a mammal, such as the mouse, has significant advantages over current protocols for gene inactivation. Because the gene silencing mechanism leaves the genomic locus intact, the silencing effect is potentially reversible. In addition, transgenic RNAi is faster and significantly less costly than current locus inactivation protocols.

Recent tissue culture studies provide strong evidence that the fundamental mechanisms for RNAi are evolutionarily conserved and operational in mammalian cells. However, the existence of the interferon response in mammalian cells is an obstacle to the implementation of dsRNA-based strategies. In a specific embodiment gene silencing is effected by expression of long interrupted RNAs, herein termed “bubble hybrid” RNAs. Although any target gene expression may be affected, in exemplary embodiments two model systems are utilized to evaluate the efficacy of gene inactivation: the inhibition of tyrosinase expression in melanocytes and in the Retinal Pigmented Epithelium (RPE), resulting in changes in pigmentation, and the inactivation of Rb in lens fiber cells, resulting in cataracts and microphthalmia. Again, RNAi technology is more flexible than traditional gene knock-out techniques and greatly accelerates the functional characterization of the mammalian genome.

In specific embodiments, any promoter may regulate expression of the transgene encoding the bubble hybrid RNA, but in specific embodiments tissue-specific promoters, ubiquitous promoters, constitutive promoters, inducible promoters, and the like could be used. Since RNAi-dependent gene suppression is post-transcriptional, the endogenous locus remains intact. Depending on the stability of the siRNA, shut-off or reduction of transgene expression may permit the resumption of synthesis of the endogenous target protein, thus providing reversible (i.e. transient) gene inhibition.

In one embodiment of the present invention, there is an RNA composition that comprises at least one double strand region, wherein said double stranded region is interrupted by at least one region of non-complementarity, wherein said composition induces destruction of a target nucleic acid sequence. The target nucleic acid sequence may be a transcript, and in some embodiments the composition is substantially incapable of eliciting an interferon pathway in a cell. The RNA composition may comprises one RNA molecule or more than one molecule. The composition may further be defined as comprising a construct, said construct having in a 5′ to 3′ orientation: a first double stranded RNA region; a first region of non-complementarity; a second double stranded RNA region; a second region of non-complementarity; and a third double stranded RNA region.

In specific embodiments, the RNA composition comprises at least one regulatory sequence operably linked to the construct, and the regulatory sequence may be a constitutive promoter, an inducible promoter, a tissue-specific promoter, or a combination thereof. In another specific embodiment, one or more double stranded RNA regions are at least about 22 nucleotides in length, between about 22 and about 30 nucleotides in length, or between about 27 and about 30 nucleotides in length. The regions of non-complementarity may be at least about 5 nucleotides in length, or from about 5 to about 12 nucleotides in length.

In other specific embodiments, the one or more double stranded RNA regions are complementary to a target nucleic acid sequence, and they can be complementary to the same target nucleic acid sequence or to different target nucleic acid sequences. In specific embodiments they are fully complementary to a target nucleic acid sequence. They may be complementary to a 5′ region of a target transcript, such as a 5′ untranslated region of a target transcript, and/or complementary to a 3′ region of a target transcript, such as a 3′ untranslated region of a target transcript.

The composition may be encoded by a single transgene or by two or more transgenes. Also, the junction between at least one double stranded RNA region and at least one region of non-complementarity comprises at least two consecutive T's, in some embodiments. The composition may further be defined as comprising n number of double stranded regions and n-1 number of regions of non-complementarity.

In other aspects of the present invention, there is a vector comprising an RNA composition described herein, a transgene comprising an RNA composition as described herein, or a mammalian cell comprising an RNA composition as described herein.

In additional embodiments, there is a transgenic, non-human animal having at least one cell comprising a transgene encoding a RNA composition as described herein, wherein the transgene is expressed in one or more cells of the transgenic animal, resulting in inducing destruction of at least one target nucleic acid sequence by the RNA composition.

In an additional embodiment of the present invention, there is an RNA composition comprising two or more double strand regions, adjacent regions of which are separated from each other by one or more regions of non-complementarity, wherein at least two of said double strand regions are complementary to at least two different target transcripts, wherein said RNA composition is capable of inducing destruction of said transcripts. The double stranded regions may be fully complementary to said transcripts.

In an additional embodiment of the present invention, there is a vector having a promoter that operably regulates sequence that encodes an RNA, wherein said sequence comprises one or more nucleic acid sequence constructs each of which are flanked by at least two restriction enzyme sites, wherein upon intramolecular hybridization of said RNA, at least one of said constructs generates a region of non-complementarity within said RNA. The two restriction enzyme sites may be non-identical. The sequence of the vector may also comprise a signal for poly (A) addition. In a specific embodiment, the sequence is further defined as having the following components present in a 5′ to 3′ orientation: a) a first restriction enzyme site; b) a second restriction enzyme site; c) sequence that encodes one strand of a first region of non-complementarity; d) a third restriction enzyme site; e) a fourth restriction enzyme site; f) sequence that encodes one strand of a second region of non-complementarity; g) a fifth restriction enzyme site; h) a sixth restriction enzyme site; i) sequence that encodes one strand of a third region of non-complementarity; j) a loop region; k) sequence that encodes a second strand of the third region of non-complementarity, wherein the sequence is non-complementary to the sequence in i); l) a seventh restriction enzyme site; m) an eighth restriction enzyme site; n) sequence that encodes a second strand of the second region of non-complementarity, wherein the sequence is non-complementary to the sequence in f); o) a ninth restriction enzyme site; p) a tenth restriction enzyme site; q) sequence that encodes a second strand of the first region of non-complementarity, wherein the sequence is non-complementary to the sequence in c); and r) sequence that directs addition of a poly A tail.

In one embodiment of the present invention, there is a kit comprising an RNA composition as described herein, and the kit may further comprises one or more restriction enzymes and/or a buffer suitable for at least one restriction enzyme. It may also comprise a RNA polymerase.

In another embodiment of the present invention, there is a eukaryotic cell exhibiting a target nucleic acid sequence-specific knockout phenotype, wherein said cell is transfected with at least one RNA composition capable of and under conditions suitable for inducing destruction of the target nucleic acid sequence, wherein the RNA composition comprises at least one double stranded region interrupted by at least one region of non-complementarity. The cell is in a eukaryotic non-human organism, in some embodiments.

In another embodiment of the present invention, there is an isolated genetic construct that is capable of inducing destruction of at least one target nucleic acid sequence in an animal cell that is transfected with said construct, wherein the genetic construct comprises nucleic acid sequence comprising or encoding a RNA composition, said RNA composition comprising: a first double strand region that is substantially identical to at least a region of a first target nucleic acid sequence; and a second double strand region that is substantially identical to at least a region of a second target nucleic acid sequence, said first and second double stranded regions separated by a region of non-complementarity, and wherein the double strand regions are under the control of at least one operable promoter. The first and second target nucleic acid sequences may be transcripts from the same gene or locus or from a different gene or locus. The first and second double stranded regions may be under the control of different operable promoters.

In additional embodiments there is a method of inducing destruction of a target nucleic acid sequence in an animal cell, comprising expressing in said animal cell a genetic construct as described herein.

In an additional embodiment of the present invention, there is a method of inducing destruction of at least one target nucleic acid sequence in a cell, comprising introducing to the cell an effective amount of a RNA composition comprising one or more double stranded RNA regions each of which are substantially identical to a portion of a target nucleic acid sequence, and each of which said double stranded regions are separated by the adjacent double stranded RNA region by a region of non-complementarity, wherein upon said introducing said RNA composition to the cell, said composition induces destruction of said target nucleic acid sequence. The cell may be in a mammal, such as a mouse.

In another embodiment of the present invention, there is a method of preparing an RNA composition as described herein, comprising the steps of: synthesizing two RNA strands, wherein said RNA strands are capable of forming a double stranded RNA molecule; and combining the synthesized RNA strands under conditions wherein a double stranded RNA molecule is produced, said double stranded RNA molecule capable of inducing destruction of a target nucleic acid sequence. The RNA strands may be chemically synthesized or enzymatically synthesized. The combining step occurs in a cell following introduction into the cell of the two RNA strands or nucleic acids encoding them, in specific embodiments.

In another embodiment of the present invention, there is a method of preparing a single stranded RNA composition of claim 1, comprising the steps of obtaining at least one region of a nucleic acid encoding said RNA composition; obtaining at least another region of a nucleic acid encoding said RNA composition; cloning said regions operably together in a vector to produce a single transgene, wherein said vector comprises at least one regulatory sequence operably linked to said transgene; and expressing said RNA composition.

In an additional embodiment of the present invention, there is a method of mediating RNA interference of a nucleic acid sequence in a cell or organism, comprising: introducing into the cell or organism at least one RNA composition, wherein the RNA composition comprises in a 5′ to 3′ direction at least: a first double stranded region; a region of non-complementarity; and a second double stranded region, wherein at least one of the double stranded regions targets the nucleic acid sequence for degradation; and maintaining the cell or organism under conditions wherein degradation of the target nucleic acid sequence occurs. In a specific embodiment, there is a nucleic acid sequence that encodes a cellular mRNA or a viral mRNA.

In another embodiment of the present invention, there is a method of inducing destruction of nucleotide sequence from more than one locus, comprising: introducing into the cell or organism at least one RNA composition, wherein the RNA composition comprises in a 5′ to 3′ direction at least: a first double stranded region; a region of non-complementarity; and a second double stranded region, wherein the double stranded regions target different nucleic acid sequences for degradation; and maintaining the cell or organism under conditions wherein the nucleotide sequences are destroyed. In a specific embodiment, there is a method is further defined as destroying a transcript from more than one gene.

In a specific embodiment, there is a method of producing double stranded RNA fragments from a longer RNA composition (which may be referred to as a “parent” RNA composition), wherein the fragments are at least about 25, 26, 27, 28, 29, 30, and so forth nucleotides in length and wherein the fragments mediate RNA interference of mRNA of a gene (which may also be referred to as nucleic acid sequence) to be degraded, wherein the parent RNA composition comprises at least one bubble separating two double stranded regions, wherein the method comprises the steps of: producing the parent RNA composition that corresponds in at least one of the double stranded regions to the nucleic acid sequence to be degraded; and providing the parent RNA composition with conditions under which the double stranded parent RNA composition is processed to double stranded RNA fragments of from about 25 nucleotides to about 30 nucleotides, wherein at least one of the fragments mediates RNA interference of the mRNA to be degraded, thus wherein said RNA interference occurs.

In another specific embodiment, the methods described herein are useful for examining the function of a particular nucleic acid sequence, such as a gene or mRNA, in a cell or organism by targeting the desired nucleic acid sequence with RNA compositions as described herein, and observing, analyzing, assaying and so forth the phenotype of the cell or organism produced thereby and, optionally, comparing the detectable phenotype to an appropriate control cell or control organism, thereby providing information about the function of the gene. In a specific embodiment, the reduction or inhibition of expression of the desired nucleic acid sequence is monitored by standard means in the art to confirm successful inhibition/reduction of its expression.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 provides sequences of the bubble-hybrid transcripts for RNAi of Rb in the mouse. Sequences correspond to base pairs 774-802, 2414-2442, and 3173-3201 of the mouse Rb cDNA sequence. Flanking restriction sites HindIII and PstI are used for directional cloning. TT dinucleotides flank the non-pairing bubbles. BglII and NheI unique sites have been engineered for identification of recombinant clones.

FIG. 2 provides a protocol to test for the formation of siRNAs in transgenic tissues.

FIG. 3 illustrates one exemplary embodiment of a bubble hybrid RNAi. Two pseudo-complementary transcripts hybridize to form a dsRNA with intercalated non-complementary linkers that form hybridization “bubbles”.

FIG. 4 illustrates one exemplary embodiment of generation of bubble hybrid transgenes. The α and β transgenes are generated by PCR using two partly overlapping polynucleotides. In specific embodiments, the oligonucleotides include restriction sites to allow directional insertion downstream of the tissue-specific promoter.

FIG. 5 illustrates an exemplary embodiment of generation of a single transgene bubble hybrid. The transgenes are generated by primer extension using two pairs of partly overlapping polynucleotides. The polynucleotides include unique restriction sites to allow assembly of the two resulting components of the transgene, and insertion downstream of the tissue-specific promoter and upstream of the SV40 intron/polyA .

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

Throughout this specification and the claims that follow, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Invention Definitions

The term “region of non-complementarity” as used herein refers to an entity in a double stranded region of an RNA composition (wherein the double strand nature of the RNA composition may arise from intramolecular hybridization within one RNA molecule and/or arise from intermolecular hybridization between two RNA molecules) that comprises non-complementary nucleotides between the two strands of the double stranded region. Thus, the region may be defined as a region of non-complementary nucleotides flanked by regions of double stranded RNA. In specific embodiments, the length of non-complementation is at least about 5 nucleotides. In other specific embodiments, the junction between the bubble and double stranded region comprises at least two T's. The term “bubble” may also be used for the term “region of non-complementarity”, and the term “bubble” implies no specific shape of said region, although in some embodiments it is shaped as a bubble.

The term “nucleic acid construct” as used herein is a nucleic acid engineered or altered by the hand of man, and generally comprises one or more nucleic acid sequences organized by the hand of man. In a specific embodiment, the construct comprises at least one restriction enzyme site, such as Eco RI, BamHI, and so forth.

The term “expressing,” “expression,” or “express” as used herein refers to the production of an mRNA transcript from a nucleic acid sequence encoding thereof. Thus, the term may be defined as referring to transcription.

The terms “destruction,” “destroy,” or “destroying” refer to the damage of a target nucleic acid sequence. In specific embodiments, they refer to the breaking of a RNA molecule into smaller fragments. In other specific embodiments, they involve enzymatic-induced destruction.

The term “gene” as used herein is to be taken in its broadest context and includes the following: a standard genomic gene consisting of transcriptional and/or translational regulatory sequences and/or a coding region and/or non-translated sequences (i.e. introns, 5′- and 3′-untranslated sequences); mRNA or cDNA corresponding to the coding regions (i.e. exons) optionally comprising 5′- or 3′-untranslated sequences linked thereto; and/or an amplified DNA fragment or other recombinant nucleic acid molecule produced in vitro and comprising all or a part of the coding region and/or 5′- or 3′-untranslated sequences linked thereto. The term “gene” is also used to describe synthetic or fusion molecules encoding all or part of a functional product, in particular a sense or antisense mRNA product or a peptide, oligopeptide or polypeptide or a biologically-active protein.

The term “synthetic gene” refers to a non-naturally occurring gene as hereinbefore defined which preferably comprises at least one or more transcriptional and/or translational regulatory sequences operably linked to a structural gene sequence.

The term “locus” as used herein refers to a position on a chromosome at which a gene for a particular gene product resides; any one of the alleles for the gene may be present at the locus.

The term “post-transcriptional” as used herein refers to events following the mechanism of transcription but prior to the mechanism of translation. Examples of post-transcriptional processes included adding of a 5′ cap, addition of a poly(A) tail, and so forth.

The term “RNA interference (RNAi)” as used herein refers to double stranded RNA-dependent interference with gene expression via specific destruction of a transcript.

The term “short interfering RNA (siRNA)” as used herein refers to an introduced or generated intermediate of target transcript destruction.

The term “structural gene” shall be taken to refer to a nucleotide sequence that is capable of being transmitted to produce mRNA and optionally, encodes a peptide, oligopeptide, polypeptide or biologically active protein molecule. Those skilled in the art will be aware that not all mRNA is capable of being translated into a peptide, oligopeptide, polypeptide or protein, for example if the mRNA lacks a functional translation start signal or alternatively, if the mRNA is antisense mRNA. The present invention clearly encompasses synthetic genes comprising nucleotide sequences that are not capable of encoding peptides, oligopeptides, polypeptides or biologically-active proteins. In particular, the present inventors have found that such synthetic genes may be advantageous in modifying target gene expression in cells, tissues or organs of a prokaryotic or eukaryotic organism.

The term “substantially incapable of eliciting an interferon response” as used herein refers to being substantially incapable of eliciting an interferon response that nonspecifically suppresses gene expression.

The term “target gene” shall be taken to refer to any gene, the expression of which is to be modified using the synthetic gene of the invention. Preferred target genes include, but are not limited to, viral genes and foreign genes that have been introduced into the cell, tissue or organ or, alternatively, genes that are endogenous to the cell, tissue, or organ.

The term “transcript” as used herein refers to an mRNA encoded by a nucleic acid sequence, such as a gene. The transcript may comprise post-transcriptional modification, such as a 5′, a poly (A) tail, an intron(s) removed by splicing, and the like.

The term “transgene” as used herein refers to any nucleic acid molecule that is inserted into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term may also refer to a nucleic acid molecule that includes one or more selected nucleic acid sequences, e.g., (DNAs) that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location that differs from that of the natural gene. A transgene may include one or more promoters and any other DNA, such as introns, necessary for expression of the selected nucleic acid sequence, all operably linked to the selected sequence, and may include an enhancer sequence.

The Present Invention

The present invention provides a strategy for inducible and reversible tissue-specific gene silencing in a mammal, such as the mouse. Successful implementation of this strategy would revolutionize the fields of mammalian functional genomics and developmental biology (Zamore, 2001).

The compositions of the present invention are designed to yield tissue-specific siRNAs that will target endogenous genes without inducing the non-specific interferon response. Given that the fundamental mechanisms for destruction of target messenger RNA by siRNAs, and/or translational interference by “short temporal antisense RNA” (stRNA), are evolutionarily conserved (Pasquinelli et al., 2001; Bosher and Labouesse, 2000; Ketting et al., 2000; Kuwabara and Coulson, 2000), the present inventors deduced the present invention by exploiting these conserved mechanisms to attain precise and reversible control of gene expression in transgenic mice. Thus, RNAi technology could supercede traditional gene knock-out techniques and would greatly accelerate the functional characterization of the mammalian genome.

The present invention is directed to an RNA composition having at least one double stranded region comprising non-complementarity in at least one part of the double stranded region, wherein the composition is effective for reducing/inhibiting transcription without eliciting an interferon pathway. In a specific embodiment, the non-complementary region is a bubble in form. It is not obvious to those in the art that a double stranded RNA comprising at least one bubble region would work in the present invention, particularly given that it is known in the art that double strand RNA comprising even only one mismatch resulted in a reduction in RNA interference (Hamada et al. (2002). The present invention overcomes such deficiencies in the art.

Thus, the present invention regards compositions and methods related to the development in a mammal such as the mouse of a gene inactivation strategy based on RNA interference (RNAi). This strategy has significant advantages over the genetic engineering techniques currently available for the manipulation of gene expression in the mouse. Evidence derived from cell culture experiments indicates that the core elements of the RNAi pathway are operational in mammalian cells (Elbashir et al., 2001; Svoboda et al., 2000; Wianny et al., 2000), and suggests that RNAi can be used to achieve tissue-specific gene silencing in transgenic mice. The present inventors in some embodiments may use two exemplary model systems that provide easy assays for inhibition of gene expression. One is inhibition of tyrosinase in melanocytes and in retinal pigmented epithelium (RPE). The other is inhibition of the expression of Rb in the lens. Suppression of tyrosinase expression causes a change in pigmentation (Yokoyama et al., 1990), while loss of Rb in lens fiber cells leads to cataracts and microphthalmia (Fromm et al., 1994).

The present invention provides that gene silencing can be accomplished by expression of long interrupted RNAs, which may be referred to as “bubble hybrid” RNAs. Given that RNAi-mediated gene silencing in C. elegans involves endonuclease-mediated processing of longer dsRNAs, the present inventors determine that transgenic production of an interrupted dsRNA (bubble hybrid) results in siRNA-mediated gene inhibition without activation of the interferon response.

In specific and exemplary embodiments, transgenic mice are generated that express unique complementary RNAs that can hybridize to form bubble hybrids to target tryrosinase and Rb. The transgenic mice are assayed for transgene expression, bubble hybrid formation and/or processing, for coat color changes or cataracts/microphthalmia, and/or for loss or reduction of target gene expression, in these exemplary embodiments.

In another aspect, the invention includes host cells, e.g., mammalian cells, that contain the new nucleic acid molecules. The invention also includes transgenes that include the new nucleic acid molecules.

In an aspect of the invention, the invention features transgenic, non- human animals, one or more of whose cells include a transgene encoding one or more of the RNA compositions described herein, wherein the transgene is expressed in one or more cells of the transgenic animal resulting in the animal exhibiting ribonucleic acid interference (RNAi) of the target gene by at least one RNA composition described herein. In these animals, the regulatory sequence for the transgene can be constitutive or inducible, or the regulatory sequence can be tissue-specific. In a specific embodiment, the RNA interference is elicited in a tissue-specific manner in a mammal, such as in a transgenic mouse. For example, the transgene can be expressed selectively in one or more cells, such as cardiac cells, lymphocytes, liver cells, vascular endothelial cells, lens cells, melanocytes, or spleen cells. In some embodiments, the regulatory sequence can be a Pol III or Pol II promoter and can be an exogenous sequence. These transgenic animals can be non-human primates or rodents, such as mice, rats, or other animals (e.g., other mammals, such as goats or cows; or birds).

The invention also includes cells derived from the new transgenic animals. For example, these cells can be a lymphocyte, a hematopoietic cell, a liver cell, a cardiac cell, a vascular endothelial cell, a lens cell, a melanocyte, or a spleen cell.

In some aspects of the present invention, the invention includes methods of inducing ribonucleic acid interference of a target nucleic acid sequence in a cell, e.g. in an animal or in culture. The new methods include obtaining a transgenic animal comprising a transgene including a nucleic acid molecule encoding a RNA composition of the present invention under the operable control of a promoter, such as an inducible promoter; and inducing the cell to express the RNA composition to ultimately form at least one small interfering ribonucleic acid (siRNA) within the cell, thereby inducing RNAi of the target nucleic acid sequence in the animal.

Alternatively, the methods include obtaining a host cell; culturing the cell; and enabling the cell to express the RNA composition to form a small interfering ribonucleic acid (siRNA) within the cell, thereby inducing RNAi of the target gene in the cell.

The next sections provide a brief overview of materials and techniques that a person of ordinary skill would deem important to the practice of the invention. These sections are followed by a more detailed description of the various embodiments of the invention.

RNA Compositions

The present invention is directed to an RNA composition having at least one double stranded region comprising non-complementarity in at least one part of the double stranded region, wherein the composition is effective for reducing/inhibiting transcription without eliciting an interferon pathway. In a specific embodiment, the non-complementary region is a bubble in form. In a specific embodiment, the double stranded bubble hybrid RNA composition is processed to smaller components, such as processing the bubble hybrid RNA to its double stranded components. In a specific embodiment, an RNA composition of the present invention is isolated.

In specific embodiments, the RNA composition comprises a single RNA molecule capable of folding back on itself through, for example, intramolecular hybridization. In other embodiments, the RNA composition comprises two or more RNA molecules having intermolecular hybridization. At least one double stranded region of these compositions comprises a bubble.

In particular embodiments, a double stranded region is at least about 22, 23, 24, 25, 26, 27, 28, 29, 30, 31, 32, or more nucleotides in length. In other specific embodiments, the bubble region is about 5, 6, 7, 8, 9, 10, or 11 nucleotides in length. In additional specific embodiments, there are at least about 2 double stranded regions in the RNA composition, and preferably 2-5 double stranded regions. Each double stranded region may comprise complementarity to different target nucleic acid sequences. In a specific embodiment, the RNA composition is about 30%, about 35%, about 40%, about 45%, about 50%, about 55%, about 60%, about 65% or about 70% GC-rich.

In some embodiments of the present invention, the terminal 3′ bases comprise hydroxyls, although in alternative embodiments they do not.

The RNA composition may comprise one or more strands of polymerized ribonucleotide. It may include modifications to either the phosphate-sugar backbone or at least one nucleoside. For example, the phosphodiester linkages of natural RNA may be modified to include at least one of a nitrogen or sulfur heteroatom, or both. Modifications in RNA structure may be tailored to allow specific genetic inhibition while avoiding a general panic response in some organisms that is generated by dsRNA. Likewise, bases may be modified to block the activity of adenosine deaminase. The RNA composition may be produced enzymatically or by partial/total organic synthesis; any modified ribonucleotide can be introduced by in vitro enzymatic or organic synthesis.

The RNA composition may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected. Physical methods of introducing nucleic, acids include injection directly into the cell or extracellular injection into the organism of an RNA solution.

The RNA structure comprising a double stranded region may be formed by a single self-complementary RNA strand or two complementary RNA strands. RNA duplex formation may be initiated either inside or outside the cell. The RNA may be introduced in an amount that allows delivery of at least one copy per cell. Higher doses (e.g., at least 5, 10, 100, 500 or 1000 copies per cell) of double stranded material may yield more effective inhibition; lower doses may also be useful for specific applications. Inhibition is sequence-specific in that nucleotide sequences corresponding to the duplex region of the RNA are targeted for genetic inhibition. RNA compositions containing nucleotide sequences identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (for example, at 400 mM NaCl, 40 mM PIPES pH 6.4, 1 mM EDTA, 50° C. or 70° C. hybridization for 12-16 hours; followed by washing). The length of the identical nucleotide sequences may be, for example, at least 20, 25, 50, 100, 200, 300 or 400 or more bases.

Although 100% sequence identity between the dsRNA and the target nucleic acid sequence is prepared, in alternative embodiments 100% sequence identity between the RNA and the target gene is not required to practice the present invention. Thus, the invention has the advantage of being able to tolerate sequence variations that might be expected due to genetic mutation, strain polymorphism, or evolutionary divergence.

The RNA composition may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, and/or polyadenylation) may be used to transcribe the RNA strand (or strands). Inhibition of a target sequence may be targeted by specific transcription in an organ, tissue, or cell type; stimulation of an environmental condition (e.g., infection, stress, temperature, chemical inducers); and/or manipulating transcription at a developmental stage or age. The RNA strand(s) may or may not be polyadenylated; the RNA strand(s) may or may not be capable of being translated into a polypeptide by a cell's translational apparatus. The RNA composition may be chemically or enzymatically synthesized by manual or automated reactions. The RNA composition may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography or a combination thereof. Alternatively, the RNA composition may be used with no or a minimum of purification to avoid losses due to sample processing.. The RNA composition may be dried for storage or dissolved in an aqueous solution. The solution may contain buffers or salts to promote annealing, and/or stabilization of the duplex strands. In some embodiments of the present invention, RNAase inhibitors are utilized in a solution comprising the RNA composition(s).

Generally, an RNA composition of the present invention or a nucleic acid sequence encoding same may be subjected to mutagenesis to produce single or multiple nucleotide substitutions, deletions and/or additions without affecting its ability to modify target gene expression. Nucleotide insertional derivatives of the synthetic gene of the present invention include 5′ and 3′ terminal fusions as well as intra-sequence insertions of single or multiple nucleotides. Insertional nucleotide sequence variants are those in which one or more nucleotides are introduced into a predetermined site in the nucleotide sequence, although random insertion is also possible with suitable screening of the resulting product.

Deletional variants may be characterized by the removal of one or more nucleocides from the sequence. Substitutional nucleotide variants may be those in which at least one nucleotide in the sequence has been removed and a different nucleotide inserted in its place. Such a substitution may be “silent” in that the substitution does not change the amino acid defined by the codon. Alternatively, substituents are designed to alter one amino acid for another similar acting amino acid, or amino acid of like charge, polarity, or hydrophobicity.

Accordingly, the present invention extends to homologs, analogs and derivatives of nucleic acid sequences encoding the RNA compositions described herein.

For the present purpose, “homologs” of an RNA composition or nucleic acid sequence encoding same as hereinbefore defined or of a nucleotide sequence shall be taken to refer to an isolated nucleic acid molecule that is substantially the same as the nucleic acid molecule of the present invention or its complementary nucleotide sequence, notwithstanding the occurrence within said sequence, of one or more nucleotide substitutions, insertions, deletions, or rearrangements.

“Analogs” of a RNA composition or nucleic acid sequence encoding same as hereinbefore defined or of a nucleotide sequence set forth herein shall be taken to refer to an isolated nucleic acid molecule that is substantially the same as a nucleic acid molecule of the present invention or its complementary nucleotide sequence, notwithstanding the occurrence of any non-nucleotide constituents not normally present in said isolated nucleic acid molecule, for example caxbohydrates, radiochemicals (including radionucleotides), reporter molecules such as, but not limited to DIG, alkaline phosphatase, or horseradish peroxidase, amongst others.

“Derivatives” of a RNA composition or nucleic acid sequence encoding same as hereinbefore defined or of a nucleotide sequence set forth herein shall be taken to refer to any isolated nucleic acid molecule that contains significant sequence similarity to said sequence or a part thereof. Generally, the nucleotide sequence encoding an RNA composition of the present invention may be subjected to mutagenesis to produce single or multiple nucleotide substitutions, deletions and/or insertions. Nucleotide insertional derivatives of the nucleotide sequence of the present invention include 5′ and 3′ terminal fusions as well as intra-sequence insertions of single or multiple nucleotides or nucleotide analogues. Insertional nucleotide sequence variants are those in which one or more nucleotides or nucleotide analogues are introduced into a predetermined site in the nucleotide sequence of said sequence, although random insertion is also possible with suitable screening of the resulting product being performed. Deletional variants are characterized by the removal of one or more nucleotides from the nucleotide sequence. Substitutional nucleotide variants are those in which at least one nucleotide in the sequence has been removed and a different nucleotide or nucleotide analogue inserted in its place.

Accordingly, a nucleic acid sequence encoding an RNA composition may comprise a nucleotide sequence that is at least about 80% identical to at least about 20 contiguous nucleotides of an endogenous target gene, a foreign target gene or a viral target gene present in a cell, tissue or organ or a homolog, analog, derivative thereof or a complementary sequence thereto.

Physical methods of introducing nucleic acids into a cell or organism include injection of a solution containing the RNA composition, bombardment by particles covered by the RNA composition, soaking the cell or organism in a solution of the RNA, or electroporation of cell membranes in the presence of the RNA composition. A viral construct packaged into a viral particle would accomplish both efficient introduction of an expression construct into the cell and transcription of a RNA composition encoded by the expression construct. Other methods known in the art for introducing nucleic acids to cells may be used, such as lipid-mediated carrier transport, chemicalmediated transport, such as calcium phosphate, and the like. Thus the RNA composition may be introduced along with components that perform one or more of the following activities: enhance RNA uptake by the cell, promote annealing of the duplex strands, stabilize the annealed strands, or otherwise facilitate or increase inhibition of the target gene.

In a further embodiment, a nucleic acid sequence encoding a RNA composition of the present invention also comprises a sequence encoding a reporter gene or sequence. For example, nucleic acid sequence encoding a RNA composition of the present invention according to this aspect of the invention comprises the coding region of a tyrosinase gene, in particular the murine tyrosinase gene, placed in the sense orientation operably under the control of the CMV IE promoter or SV40 late promoter. As with other embodiments described herein, the gene (i.e. tyrosinase gene) may lack a functional translation start site or be introduced in the antisense orientation. The present invention clearly encompasses all such embodiments.

As used herein, the term “tyrosinase gene” shall be taken to refer to a structural gene, cDNA molecule, genomic gene or nucleotide sequence which is capable of encoding the tyrosinase enzyme or a polypeptide fragment thereof or alternatively, a nucleotide sequence which is complementary to said structural gene, cDNA molecule, genomic gene or nucleotide sequence. Particularly preferred tyrosinase genes for use in the performance of the present invention include, but are not limited to, those described by Kwon et al. (1988) and homologues, analogues and derivatives thereof and complementary nucleotide sequences thereto.

In still a further alternative embodiment, the nucleic acid sequence encoding a RNA composition of the present invention according to this aspect of the invention comprises the coding region of the lacI gene, placed in the sense orientation operably under the control of the CMV IE promoter or SV40 late promoter. As with other embodiments described herein, the synthetic gene (i.e. E. coli lacI gene) may lack a functional translation start site or be introduced in the antisense orientation. The present invention clearly encompasses all such embodiments.

As used herein, the term “lacI gene” shall be taken to refer to a structural gene, cDNA molecule, genomic gene or nucleotide sequence that is capable of encoding a polypeptide repressor of the lacZ gene which encodes the enzyme beta-galactosidase or alternatively, a nucleotide sequence that is complementary to said structural gene, cDNA molecule, genomic gene or nucleotide sequence. Those skilled in the art will be aware that the lac repressor is a DNA-binding protein which acts on the lac operator-promoter sequence. In the presence of one of a variety of beta.-galactosides, the affinity of the lac repressor for the lac operator-promoter sequence is lowered, thereby allowing RNA polymerase to bind the lac operator-promoter region to activate transcription of the lac operon.

Target Nucleic Acids and Genes

The present invention regards the inhibition or reduction of expression of a nucleic acid sequence, such as a gene, targeted by a RNA composition of the present invention.

Wherein the target gene is a viral gene, it is particularly preferred that the viral gene encodes a function that is essential for replication or reproduction of the virus, such as but not limited to a DNA polymerase or RNA polymerase gene or a viral coat protein gene, amongst others. In a particularly preferred embodiment, the target gene comprises an RNA polymerase gene derived from a single-stranded (+) RNA virus such as bovine enterovirus (BEV), Sinbis alphavirus or a lentivirus such as, but not limited to, an immunodeficiency virus (e.g. HIV-1) or alternatively, a DNA polymerase derived from a double-stranded DNA virus such as bovine herpes virus or herpes simplex virus I (HSVI), amongst others.

Wherein the target gene is a foreign gene, those skilled in the art will be aware that it will have been introduced to the cell, tissue or organ using transformation technology or, alternatively, comprise a gene derived from a pathogen that has been introduced to said cell, tissue or organ by naturally-occurring or non-naturally occurring gene transfer processes. Particularly preferred foreign target genes include any transgene which has been introduced to the cell, tissue or organ.

Wherein the target gene is a gene that is endogenous to the cell, tissue or organ, it is particular preferred that its expression is capable of being monitored, such as by a visual assay, enzyme assay or immunoassay. Particularly preferred endogenous target genes are those detected by visual assay means.

The synthetic genes of the present invention may be derived from naturally-occurring genes by standard recombinant techniques, the only requirement being that the synthetic gene is substantially identical at the nucleotide sequence level to at least a part of the target gene, the expression of which is to be modified. By “substantially identical” is meant that the structural gene sequence of the synthetic gene is at least about 80%-90% identical to 20 or more contiguous nucleotides of the target gene, more preferably at least about 90-95% identical to 20 or more contiguous nucleotides of the target gene more preferably at least about 95-99% identical, and even more preferably absolutely identical to 20 or more contiguous nucleotides of the target gene.

The region of a gene that is targeted may be a coding region or a non-coding region. It may be a promoter, 5′ untranslated region, an exon, an intron, a 3′ untranslated region, or a combination thereof. The targeted site(s) may be in the 5′ part of the gene or in the 3′ part of the gene. A skilled artisan recognizes that the present invention is particularly well-suited to identifying regions of a gene that are efficient for targeting. For example, in specific embodiments the present invention allows targeting of multiple regions of a gene, which in some animals is considerable distance between one part of a gene and another, and so a comparison may be made between the multiple regions.

As disclosed herein, the present invention is not limited to any type of target gene or nucleotide sequence. But the following classes-of possible target genes are listed for illustrative purposes: developmental genes (e.g., adhesion molecules, cyclin kinase inhibitors, Writ family members, Pax family members, Winged helix family members, Hox family members, cytokines/lymphokines and their receptors, growth/differentiation factors and. their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, Rubiscos, topoisomerases, and xylanases).

In a specific embodiment, the target gene is a pathogen-associated gene, a viral gene, a tumor-associated gene, an autoimmune disease-associated gene, or any gene for which it would be desirable to reduce or inhibit expression. In particular embodiments the target sequences are mammalian genes, such as, but not limited to Erb-B2, APPBP2, BMP7, CCNDL, CRYM, ERL, FKBP5, FLJ20940, GRB7, HOXB7, LM04, MCC9753, MLN64, MYBL2, MYC, NBSI, NCOA3, PIPSK2B, PNMT, PPARBP, PPMID, RADSIC, RAEI, RPS6K. S100P, TBX2, TMEPAI, TRIM37, TXNIP, and ZNF217. Many of these gene targets are very important drug targets , since it is known that they are involved in mediating cancers, such as specific breast or prostate cancers.

The target gene may be a gene derived from the cell, an endogenous gene, a transgene, or a gene of a pathogen that is present in the cell after infection thereof.

Depending on the particular target gene and the dose of double stranded RNA material delivered, the procedure may provide partial or complete loss of function for the target gene. Lower doses of injected material and longer times after administration of the RNA composition of the present invention may result in inhibition in a smaller fraction of cells. Quantitation of gene expression in a cell may show similar amounts of inhibition at the level of accumulation of target mRNA or translation of target protein.

“Inhibition of gene expression” refers to the absence or detectable decrease in the level of protein and/or mRNA product from a target gene. “Specificity” refers to the ability to inhibit the target gene without manifest effects on other genes of the cell. The consequences of inhibition can be confirmed, for example, by examination of outward properties of the cell or organism (such as are described herein) or by biochemical techniques, such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and/or fluorescence activated cell analysis (FACS), which are all well-known procedures in the art. For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta-glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and/or tetracyclin.

Depending on the assay, quantitation of the amount of gene expression allows one to determine a degree of inhibition that is greater than about 5%, about 10%, about 20%, about 25% about 33%, about 45% about 50%, about 55%, about 60%, about 65%, about 70%, about 75%, about 80%, about 85% about 90%, about 95% or about 99%, or about 100% as compared to a cell not treated according to the present invention. Lower doses of injected material and longer times after administration of the RNA composition may result in inhibition in a smaller fraction of cells (e.g., at least about 10%, about 20%, about 50%, about 75%, about 90%, or about 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory RNA comprising double-strandedness, or translated polypeptide may be detected with an antibody raised against the polypeptide sequence of that region.

A skilled artisan recognizes that a target nucleic acid sequence is easily identified by accessing publicly available databases, such as the National Center for Biotechnology Information's GenBank database. In an exemplary embodiment, a target nucleic acid sequence comprises SEQ ID NO:12 (GenBank Accession Number M24560), which encodes murine tyrosinase.

In some embodiments of the present invention, the construct comprising the nucleic acid sequence that encodes the RNA composition is cloned into an intron so that upon splicing the RNA composition is devoid of poly A.

Cell or Organism for Delivery of RNA Composition

The cell with the target gene may be derived from or contained in any organism (e.g., plant, animal, protozoan, virus, bacterium, or fungus). The plant may be a monocot, dicot or gymnosperm; the animal may be a vertebrate or invertebrate. The RNA composition may be synthesized either in vivo or in vitro. Endogenous RNA polymerase of the cell may mediate transcription in vivo, or cloned RNA polymerase can be used for transcription in vivo or in vitro. For generating double stranded transcripts from a transgene in vivo, a regulatory region may be used to transcribe the RNA strand (or strands).

Furthermore, genetic manipulation by the present invention becomes possible in organisms that are not classical genetic models. Breeding and screening programs may be accelerated by the ability to rapidly assay the consequences of a specific, targeted gene disruption. Gene disruptions may be used to discover the function of the target gene, to produce disease models in which the target gene is involved in causing or preventing a pathological condition, and/or to produce organisms with improved economic properties.

Preferred microbes are those used in agriculture or by industry, and those that are pathogenic for plants or animals. Fungi include organisms in both the mold and yeast morphologies.

Plants include Arabidopsis; field crops (e.g., alfalfa, barley, bean, com, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

Examples of vertebrate animals include fish and/or mammal, (cattle, goat, pig, sheep, rodent, hamster, mouse, rat, primate, and human). Invertebrate animals include nematodes, other worms, drosophila, and other insects. Representative generae of nematodes include those that infect animals (e.g., Ancylostoma, Ascaridia, Ascaris, Bunostomum, Caenorhabditis, Capillaria, Chabertia, Cooperia, Dictyocaulus, Haemonchus, Heterakis, Nematodirus, Oesophagostomum, Ostertagia, Oxyuris, Parascaris, Strongylus, Toxascaris, Trichuris, Trichostrongylus, Tflichonema, Toxocara, Uncinaria) and those that infect plants (e.g., B ursaphalenchus, Criconerriella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus, and Xiphinema. Representative orders of insects include Coleoptera, Diptera, Lepidoptera, and Homoptera.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Promoters

The RNA composition of the present invention clearly encompasses nucleic acid sequence operably connected in the sense or antisense orientation to a promoter sequence. Reference herein to a “promoter” is to be taken in its broadest context and may include the transcriptional regulatory sequences of a classical genomic gene, including the TATA box which is required for accurate transcription initiation in eukaryotic cells, with or without a CCAAT box sequence and additional regulatory elements (i.e. upstream activating sequences, enhancers and silencers). For expression in prokaryotic cells, such as bacteria, the promoter should at least contain the -35 box and -10 box sequences.

A promoter is usually, but not necessarily, positioned upstream or 5′, of the nucleic acid sequence encoding the RNA composition of the invention, the expression of which it regulates. In the present context, the term “promoter” is also used to describe a synthetic or fusion molecule, or derivative that confers, activates or enhances expression of an isolated nucleic acid molecule, in a cell, such as a plant, animal, insect, fungal, yeast or bacterial cell. Preferred promoters may contain additional copies of one or more specific regulatory elements, to further enhance expression the sequence which expression it regulates and/or to alter the spatial expression and/or temporal expression of same. For example, regulatory elements that confer inducibility on the expression of the RNA composition may be placed adjacent to a heterologous promoter sequence driving expression of a nucleic acid molecule.

Placing a nucleic acid sequence encoding an RNA composition of the present invention under the regulatory control of a promoter sequence means positioning said molecule such that expression is controlled by the promoter sequence. Promoters are generally positioned 5′ (upstream) to the sequences that they control. In the construction of heterologous promoter/nucleic acid sequence combinations it is generally preferred to position the promoter at an operable distance from the transcription start site, such as one that is approximately the same as the distance between that promoter and the sequence it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Examples of promoters suitable for use in the synthetic genes of the present invention include viral, fungal, bacterial, animal and plant derived promoters capable of functioning in plant, animal, insect, fungal, yeast or bacterial cells. The promoter may regulate the expression of the nucleic acid sequence encoding an RNA composition of the present invention constitutively, or differentially with respect to cell, the tissue or organ in which expression occurs or, with respect to the developmental stage at which expression occurs, or in response to external stimuli such as physiological stresses, or pathogens, or metal ions, amongst others.

Preferably, the promoter is capable of regulating expression of a nucleic acid molecule in a eukaryotic cell, tissue or organ, at least during the period of time over which the target nucleic acid sequence is expressed therein and more preferably also immediately preceding the commencement of detectable expression of the target gene in said cell, tissue or organ. Accordingly, strong constitutive promoters are particularly preferred for the purposes of the present invention or promoters which may be induced by virus infection or the commencement of target gene expression. Examples of preferred promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter and the like.

Particularly preferred promoters contemplated herein include promoters operable in eukaryotic cells, for example the SV40 early promoter, SV40 late promoter or the CMV IE promoter sequence. Those skilled in the art will readily be aware of additional promoter sequences other than those specifically described.

In the present context, the terms “in operable connection with” or “operably under the control” or similar shall be taken to indicate that expression of the structural gene is under the control of the promoter sequence with which it is spatially connected; in a cell, tissue, organ or whole organism.

Use of Tyrosinase Inactivation as an Index of RNAi Efficacy in the Exemplary Model

A reporter system is utilized that allows easy identification of partial reduction of target gene expression. One exemplary system, developed by an inventor of the present invention, is based on the expression of tyrosinase (Yokoyama et al., 1990). Tyrosinase is the first enzyme in the pathway that synthesizes melanin in melanocytes. Albinism in laboratory mice is caused by a mutation in their tyrosinase gene. In mice that are rescued with a tyrosinase minigene, the relative level of tyrosinase expression determines the amount of pigment (melanin) that is present in the mouse skin and hair. Because simple visual inspection allows the detection of subtle differences in coat color, minor variations in the level of tyrosinase activity can be monitored easily. Hence, coat color is utilized as a sensitive and inexpensive index of the efficacy of RNAi-mediated silencing.

Therapeutic Applications

In a specific embodiment, the present invention comprises a method for treating a mammal with an RNA-based (either directly based or indirectly based) disorder or disease by administering to the mammal a RNA composition of the present invention for initiating inhibition or reduction of expression of a target nucleic acid sequence at the mRNA level. In particular embodiments, the method comprises using RNAi to achieve transcriptional post-transcriptional gene silencing. In a specific embodiment, the mammal is a human.

In other specific embodiments, there is a method of treating a disease or condition associated with the presence of a protein in an individual comprising administering to the individual an RNA composition as described herein, wherein the RNA composition inhibits or reduces expression of a target nucleic acid sequence encoding said protein. In a specific embodiment, the target nucleic acid sequence is an mRNA.

In other embodiments, there are methods of producing knockdown cells comprising introducing into the cells RNA compositions of the present invention, wherein the cells comprise a gene desirable to be knocked down, wherein the RNA compositions comprise double stranded regions and regions of non-complementarity (in specific embodiments, these regions are about 5 to about 10 nucleotides in length), wherein said RNA compositions or double stranded RNA fragments thereof mediate said knockdown. In a specific embodiment, the processing from RNA having double stranded regions into fragments comprising double stranded sequence is facilitated, mediated, produced by, or otherwise caused by said fragments.

Lens-specific Inactivation of Rb

In another embodiment, RNAi is utilized to inhibit endogenous gene expression in the eye. The lens offers a model system to test for specific and non-specific effects of dsRNA expression. In the lens fiber cells, loss of Rb is sufficient to allow cell cycle re-entry, the onset of DNA replication and the induction of p21 transcription (Fromm et al., 1994; Morgenbesser et al., 1994; Liegeois et al., 1996; McCaffrey et al., 1999). In addition, non-specific effects including the induction of interferon can be monitored by analysis of cataract formation without induction of DNA replication (Egwuagu et al., 1994; Li et al., 1999).

Pharmaceutical Preparations

The results presented herein indicate that small dsRNAs are useful for triggering RNAi-like responses that can be utilized as both functional genomics and therapeutic tools. Thus, the present disclosure includes methods of using RNA compositions of the present invention as a treatment for disease. In a particular embodiment, the RNA composition comprises a dsRNA region corresponding to an oncogene, and as such the treatment is a treatment of a hyper-proliferative disease or disorder, such as cancer, in a subject.

The method includes administering an RNA composition or a DNA composition encoding therefore, or more than one RNA composition, or a combination of a RNA composition (or more than one) and one or more other pharmaceutical agents, to the subject in a pharmaceutically compatible carrier and in an amount effective to inhibit the development or progression of a disease. Although the treatment can be used prophylactically in any patient, such as one in a demographic group at significant risk for such diseases, subjects can also be selected using more specific criteria, such as a definitive diagnosis of the disease/condition or identification of one or more factors that increase the likelihood of developing such disease (e.g., a genetic, environmental, or lifestyle factor).

Various delivery systems are known and can be used to administer the RNA compositions as therapeutics. Such systems include, for example, encapsulation in liposomes, microparticles, microcapsules, recombinant cells capable of expressing the therapeutic molecule (s) (see, e.g., Wu et J. Biol. Chem. 262,4429, 1987), construction of a therapeutic nucleic acid as part of a retroviral or other vector, and the like. Methods of introduction include, but are not limited to, intrathecal, intradermal, intramuscular, intraperitoneal (ip), intravenous (iv), subcutaneous, intranasal, epidural, and oral routes. The therapeutics may be administered by any convenient route, including, for example, infusion or bolus injection, topical, absorption through epithelial or mucocutaneous linings (e.g., oral mucosa, rectal and intestinal mucosa, and the like) ophthalmic, nasal, and transdermal, and may be administered together with other biologically active agents. Administration can be systemic or local. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed (e.g., by an inhaler or nebulizer), for instance using a formulation containing an aerosolizing agent.

In a specific embodiment, it may be desirable to administer the pharmaceutical compositions of the invention locally to the area in need of treatment. This may be achieved by, for example, and not by way of limitation, local infusion or perfusion during surgery, topical application (e.g., wound dressing), injection, catheter, suppository, or implant (e.g., implants formed from porous, non-porous, or gelatinous materials, including membranes, such as sialastic membranes or fibers), and the like. In one embodiment, administration can be by direct injection at the site (or former site) of a tissue that is to be treated. In another embodiment, the therapeutic are delivered in a vesicle, in particular liposomes (see, e.g., Langer, Science 249,1527, 1990; Treat et al., in Liposomes in the Therapy of Infectious Disease and Cancer, Lopez-Berestein and Fidler (eds. ), Liss, N. Y., pp. 353-365,1989).

In yet another embodiment, the therapeutic can be delivered in a controlled release system. In one embodiment, a pump may be used (see, e.g., Langer Science 249,1527, 1990; Sefton Crit. Rev. Biomed. Eng. 14, 201,1987; Buchwald et al., Surgery 88,507, 1980; Saudek et al., New. Engl. J. Med. (321,) 574,1989). In another embodiment, polymeric materials can be used (see, e.g., Ranger et al., Macromol. Sci. Rev. Macromol. Chem.. 23,61, 1983; Levy et al., Science 228,190, 1985; During et al., Ann. Neurol. 25,351, 1989; Howard et al., J. Neurosurg. 71,105, 1989). Other controlled release systems, such as those discussed in the review by Langer (Science 249,1527 1990), can also be used.

The vehicle in which the agent is delivered can include pharmaceutically acceptable compositions of the compounds, using methods well known to those with skill in the art. For instance, in some embodiments, small dsRNAs typically are contained in a pharmaceutically acceptable carrier. The term “pharmaceutically acceptable” means approved by a regulatory agency of the federal or a state government or listed in the U.S. Pharmacopoeia or other generally recognized pharmacopoeia for use in animals, and, more particularly, in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions, blood plasma medium, aqueous dextrose, and glycerol solutions can also be employed as liquid carriers, particularly for injectable solutions. The medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic, if desired, can also contain minor amounts of wetting or emulsifying agents, or pH buffering agents. These therapeutics can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. A more complete explanation of parenteral pharmaceutical carriers can be found in Remington: The Science and Practice of pharmacy (19^{th} Edition, 1995), Chapter 95. Embodiments of other pharmaceutical compositions are prepared with conventional pharmaceutically acceptable counter-ions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

The composition of this invention can be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to administration.

The amount of the therapeutic that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each patient's circumstances. An example of such a dosage range is 0.1 to 200 mg/kg body weight in single or divided doses. Another example of a dosage range is 1.0 to 100 mg/kg body weight in single or divided doses.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

The RNA compositions of the present invention can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some embodiments long-term treatment with the drug is contemplated, for instance in order to reduce the occurrence of expression or overexpression of the target gene.

In some embodiments, sustained intra-tumoral (or near-tumoral) release of the pharmaceutical preparation that comprises a therapeutically effective amount of RNA composition may be beneficial. Slow-release formulations are known to those of ordinary skill in the art. By way of example, polymers such as bis (p-carboxyphenoxy) propane-sebacic acid or lecithin suspensions may be used to provide sustained intra-tumoral release.

It is specifically contemplated in some embodiments that delivery is via an injected and/or implanted drug depot, for instance comprising multi-vesicular liposomes such as in DepoFoam (SkyePharma, Inc, San Diego, Calif.) (see, for instance, Chamberlain et al, Arch. Neuro. 50: 261-264, 1993; Katri et al, J. Pharm. Sci. 87: 1341-1346,1998; Ye et al, J. Control Release 64: 155-166, 2000; and Howell, Cancer J. 7: 219-227, (2001).

In other embodiments, perfusion of a tumor with a pharmaceutical composition that contains a therapeutically effective amount of an RNA composition of the present invention is contemplated, for instance an amount sufficient to provide a measurable reduction in tumor growth, tumor size, tumor cell growth, or another measurable reduction in the disease being treated.

Combination Therapy

The present disclosure also contemplates combinations of RNA compositions with one or more other agents useful in the treatment of a disease, such as a hyper-proliferative disease. For example, RNA compositions of the present invention may be administered in combination with effective doses of other medicinal and pharmaceutical agents. In some embodiments, one or more known anti-cancer drugs are included with a RNA composition that targets a gene known to be involved in a hyper-proliferative disorder. The term “administration in combination with” refers to both concurrent and sequential administration of the active agents.

In addition, and in the exemplary therapeutic embodiment of cancer, RNA compositions may be administered in combination with effective doses of radiation, anti-proliferative agents, anti-cancer agents, immunomodulators, anti-inflammatories, anti-infectives, hypomethylation agents, nucleosides and analogs thereof, and/or vaccines.

Examples of anti-proliferative agents that can be used in combination with a dsRNA (such as an RNA compositions specific for an oncogene) include, but are not limited to, the following: ifosamide, cisplatin, methotrexate, procarizine, etoposide, BCNU, vincristine, vinblastine, cyclophosphamide, gencitabine, 5-fluorouracil, paclitaxel, and/or doxorubicin.

Non-limiting examples of immuno-modulators that can be used in combination with a RNA composition of the present invention include AS-101 (Wyeth-Ayerst Labs.), bropirimine (Upjohn), gamma interferon (Genentech), GM-CSF (granulocyte macrophage colony stimulating factor; Genetics Institute), [IL-2] (Cetus or Hoffman-LaRoche), human immune globulin (Cutter Biological), Imreg (from Imreg of New Orleans, La.), SK&F 106528, and TNF (tumor necrosis factor; Genentech).

Specific examples of particular compounds that in some embodiments are used in combination with a dsRNA (such as an dsRNA specific for ErbB2) are 5-azacytidine, 2′-deoxy-4-azacytidine, ara- C, and tricostatin A.

The combination therapies are, of course, not limited to the lists provided in these examples, but includes any composition for the treatment of diseases or conditions to which the RNA composition is targeted.

Transgenic Animals

RNA compositions of the invention can be expressed in transgenic animals. These animals may be utilized as research tools, such as for the purpose of identifying gene function, or they may represent a model system for the study of disorders that are caused by, or exacerbated by, overexpression or underexpression (as compared to wild- type or normal) of nucleic acids (and their encoded polypeptides) targeted for destruction by the RNA compositions, and for the development of therapeutic agents that modulate the expression or activity of nucleic acids or polypeptides targeted for destruction.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). Invertebrates such as Caenorhabditis elegans or Drosophila can be used as well as non-mammalian vertebrates such as fish (e.g., zebrafish) or birds (e.g. chickens).

A transgenic founder animal can be identified based upon the presence of a transgene that encodes the new RNA compositions in its genome, and/or expression of the transgene in tissues or cells of the animals, for example, using PCR or Northern analysis. Expression may be confirmed by a decrease in the expression (RNA or protein) of the target sequence.

A transgenic founder animal can be used to breed additional animals carrying the transgene. Moreover, transgenic animals carrying a transgene encoding the RNA compositions can further be bred to other transgenic animals carrying other transgenes. In addition, cells obtained from the transgenic founder animal or its offspring can be cultured to establish primary, secondary, or immortal cell lines containing the transgene.

Procedures for Making Transgenic, Non-Human Animals

A number of methods have been used to obtain transgenic, non-human animals, which are animals that have gained an additional gene by the introduction of a transgene into their cells (e.g., both the somatic and/or germ cells), or into an ancestor's germ line. In some cases, transgenic animals can be generated by commercial facilities (e.g., The Transgenic Drosophila Facility at Michigan State University, The Transgenic Zebrafish Core Facility at the Medical College of Georgia (Augusta, Ga.), and Xenogen Biosciences (St. Louis, Mo.). In general, the construct containing the transgene is supplied to the facility for generating a transgenic animal.

Methods for generating transgenic animals include introducing the transgene into the germ line of the animal. One method is by microinjection of a gene construct into the pronucleus of an early stage embryo (e.g., before the four-cell stage; Wagner et al., 1981, Proc. Natl . Acad. Sci. USA 78:5016; Brinster et al,. 1985, Proc. Natl. Acad. Sci. USA 82:4438). Alternatively, the transgene can be introduced into the pronucleus by retroviral infection. A detailed procedure for producing such transgenic mice has been described (see e.g., Hogan et al., Manipulating the Mouse Embryo, Cold Spring Harbor Laboratory, Cold Spring Harbor, N.Y. (1986); U.S. Pat. No. 5,175,383 91992). This procedure has also been adapted for other animal species (e.g. Hammer et al., 1985, Nature 315:680; Murray et al., 1989, Reprod. Fert. Devl. 1: 147; Pursel et al., 1987, Vet. Immunol. Histopath. 17: 303; Rexroad et al., 1990, J. Reprod. Fert. 41 (suppl): 119; Rexroad et al., 1989, Molec. Reprod. Devl. 1: 164; Simons et al., 1988, BioTechnology 6:179; Vize et al., 1988, J. Cell. Sci. 90:295; and Wagner, 1989, J. Cell. Biochem. 13B (suppl):164).

In brief, the procedure involves introducing the transgene into an animal by microinjecting the construct into the pronuclei of the fertilized mammalian egg (s) to cause one or more copies of the transgene to be retained in the cells of the developing mammal (s). Following introduction of the transgene construct into the fertilized egg, the egg may be incubated in vitro for varying amounts of time, or reimplanted a in surrogate host, or both. One common method is to incubate the embryos in vitro for about 1-7 days, depending on the species, and then reimplant them into the surrogate host. The presence of the transgene in the progeny of the transgenically manipulated embryos can be tested by Southern blot analysis of a segment of tissue.

Another method for producing germ-line transgenic animals is through the use of embryonic stem (ES) cells. The gene construct can be introduced into embryonic stem cells by homologous recombination (Thomas et al,. 1987, Cell 51:503; Capecchi, Science 1989, 244:1288; Joyner et al., 1989, Nature 338:153) in a transcriptionally active region of the genome. A suitable construct can also be introduced into embryonic stem cells by DNA-mediated transfection, such as by electroporation (Ausubel et al., Current Protocols in Molecular Biology, John Wiley & Sons, 1987). Detailed procedures for culturing embryonic stem cells (e.g., ES-D3, ATCC# CCL-1934, ES-E14TG2A, ATCC# CCL-1821, American Type Culture Collection, Rockville, Md.) and methods of making transgenic animals from embryonic stem cells can be found in Teratocarcinomas and Embryonic Stem Cells, A Practical Approach, ed. E. J. Robertson (IRL Press, 1987). In brief, the ES cells are obtained from pre-implantation embryos cultured in vitro (Evans et al., 1981, Nature 292: 154-156). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal.

In the above methods, the transgene can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al., 1995, Proc. Natl. Acad. Sci. USA 92: 1292). A plasmid is a DNA molecule that can replicate autonomously in a host.

The transgenic, non-human animals can also be obtained by infecting or transfecting either in vivo (e.g., direct injection), ex vivo (e.g., infecting the cells outside the host and later reimplanting), or in vitro (e.g., infecting the cells outside the host) for example, with a recombinant viral vector carrying a gene encoding the engineered RNA precursors. Examples of suitable viral vectors include recombinant retroviral vectors (Valerio et al., 1989, Gene 84:419; Scharfman et al., 1991, Proc. Natl. Acad. Sci. USA 88: 462; Miller and Buttimore, 1986, Mol. Cell. Biol. 6: 2895), recombinant adenoviral vectors (Friedman et al., 1986, Mol. Cell. Biol. 6:3791; Levrero et al., (1991,) Gene 101: 195, and recombinant Herpes simplex viral vectors (Fink et al,. 1992, Human Gene Therapy 3:11). Such methods are also useful for introducing constructs into cells for uses other than generation of transgenic animals.

Other approaches include insertion of transgenes encoding the RNA compositions into viral vectors including recombinant adenovirus, adeno- associated virus, and herpes simplex virus-1, or recombinant bacterial or eukaryotic plasmids. Viral vectors transfect cells directly. Other approaches include delivering the transgenes, in the form of plasmid DNA, with the help of, for example, cationic liposomes (lipofectin) or derivatized (e.g. antibody conjugated) polylysine conjugates, gramacidin S, artificial viral envelopes, or other such intracellular carriers, as well as direct injection of the transgene construct or CaPO₄ precipitation carried out in vivo. Such methods can also be used in vitro to introduce constructs into cells for uses other than generation of transgenic animals.

Retrovirus vectors and adeno-associated virus vectors can be used as a recombinant gene delivery system for the transfer of exogenous genes in vivo or in vitro. These vectors provide efficient delivery of genes into cells, and the transferred nucleic acids are stably integrated into the chromosomal DNA of the host. The development of specialized cell lines (termed“packaging cells”) which produce only replication-defective retroviruses has increased the utility of retroviruses for gene therapy, and defective retroviruses are characterized for use in gene transfer for gene therapy purposes (for a review see Miller, 1990, Blood 76: 271). A replication- defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9. 14 and other standard laboratory manuals.

Examples of suitable retroviruses include pLJ, pZIP, pWE and pEM which are known to those skilled in the art. Examples of suitable packaging virus lines for preparing both ecotropic and amphotropic retroviral systems include Psi-Crip, Psi- Cre, Psi-2 and Psi-Am. Retroviruses have been used to introduce a variety of genes into many different cell types, including epithelial cells, in vitro and/or in vivo (see for example Eglitis, et al,. 1985, Science 230:1395-1398; Danos and Mulligan, 1988, Proc. Natl. Acad. Sci. USA 85:6460-6464; Wilson et al, 1988, Proc. Natl. Acad. Sci. USA 85:3014-3018; Armentano et al,. 1990, Proc. Natl. Acad. Sci. USA 87:6141-6145; Huber et al., 1991, Proc. Natl. Acad. Sci. USA 88:8039-8043; Ferry et al,. 1991, Proc. Natl. Acad. Sci. USA 88:8377-8381; Chowdhury et al., 1991, Science 254:1802-1805; van Beusechem et al., 1992, Proc. Natl. Acad. Sci. USA 89:7640-7644; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In another example, recombinant retroviral vectors capable of transducing and expressing genes inserted into the genome of a cell can be produced by transfecting the recombinant retroviral genome into suitable packaging cell lines such as PA317 and Psi-CRIP (Cornette et al., 1991, Human Gene Therapy 2:5-10; Cone et al,. 1984, Proc. Natl. Acad. Sci. USA 81: 6349). Recombinant adenoviral vectors can be used to infect a wide variety of cells and tissues in susceptible hosts (e.g., rat, hamster, dog and chimpanzee) (Hsu et al., 1992, J. Infectious Disease, 166:769), and also have the advantage of not requiring mitotically active cells for infection.

Another viral gene delivery system useful in the present invention also utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988, BioTechniques 6: 616), Rosenfeld et al. (1991, Science 252: 431-434), and Rosenfeld et al. (1992, Cell 68: 143-155). Suitable adenoviral vectors derived from the adenovirus strain Ad type 5 [DL324] or other strains of adenovirus (e.g Ad2, Ad3, Ad7, etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al., 1992, cited supra). Furthermore, the virus particle is relatively stable and amenable to purification and concentration, and as above, can be modified to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham, 1986, J. Virol. 57: 267).

Yet another viral vector system useful for delivery of the subject transgenes is the adeno-associated virus (AAV). Adeno-associated virus is a naturally occurring defective virus that requires another virus, such as an adenovirus or a herpes virus, as a helper virus for efficient replication and a productive life cycle. For a review, see Muzyczka et al. (1992, Curr. Topics in Micro. and Immunol. 158 : 97-129). It is also one of the few viruses that may integrate its DNA into non-dividing cells, and exhibits a high frequency of stable integration (see for example Flotte et al. (1992, Am. J. Respir. Cell. Mol. Biol. 7:349-356; Samulski et al., 1989, J. Virol. 63:3822-3828; and McLaughlin et al. (1989, J. Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA [8 1]: 6466-6470; Tratschin et al. (1985) Mol. Cell. Biol. 4: 2072-2081; Wondisford et al. (1988) Mol. Endocrinol. 2: 32-39; Tratschin et al. (1984) J. Virol. 51: 611-619; and Flotte et al. (1993) J. Biol. Chem. 268: 3781-3790).

In addition to viral transfer methods, such as those illustrated above, non-viral methods can also be employed to cause expression of an engineered RNA precursor of the invention in the tissue of an animal. Most non-viral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene of the invention by the targeted cell. Exemplary gene delivery systems of this type include liposomal derived systems, poly-lysine conjugates, and artificial viral envelopes. Other embodiments include plasmid injection systems such as are described in Meuli et al., (2001) J. Invest. Dermatol., 116(1):131-135; Cohen et al., (2000) Gene Ther., 7(22):1896-905; and Tam et al., (2000) Gene Ther., 7(21):1867-74.

In a representative embodiment, a gene encoding an engineered RNA precursor of the invention can be entrapped in liposomes bearing positive charges on their surface (e.g. lipofectins) and (optionally) which are tagged with antibodies against cell surface antigens of the target tissue (Mizuno et al., (1992) No Shinkei Geka, 20:547-551; PCT publication W091/06309; Japanese patent application 1047381; and European patent publication EP-A-43075).

Clones of Transgenic Animals

Clones of the non-human transgenic animals described herein can be produced according to the methods described in Wilmut et al. ( (1997) Nature, 385: 810-813) and PCT publication Nos. WO 97/07668 and WO 97/07669. In brief, a cell, e.g. a somatic cell from the transgenic animal, can be isolated and induced to exit the growth cycle and enter the Gₒ phase to become quiescent. The quiescent cell can then be fused, e.g., through the use of electrical pulses, to an enucleated oocyte from an animal of the same species from which the quiescent cell is isolated. The reconstructed oocyte is then cultured such that it develops into a morula or blastocyte and is then transferred to a pseudopregnant female foster animal. Offspring borne of this female foster animal will be clones of the animal from which the cell, e.g., the somatic cell, was isolated.

Once the transgenic animal is produced, cells of the transgenic animal and cells from a control animal are screened to determine the presence of an RNA composition of the present invention, e.g., using polymerase chain reaction (PCR). Alternatively, the cells can be screened to determine if the RNA precursor is expressed (e.g., by standard procedures such as Northern blot analysis or reverse transcriptase-polymerase chain reaction (RT-PCR); Sambrook et al., Molecular Cloning-A Laboratory Manual, (Cold Spring Harbor Laboratory, 1989)).

The transgenic animals of the present invention can be homozygous or heterozygous, and one of the benefits of the invention is that the target mRNA is effectively degraded even in heterozygotes. The present invention provides for transgenic animals that carry a transgene of the invention in all their cells, as well as animals that carry a transgene in some, but not all of their cells. That is, the invention provides for mosaic animals. The transgene can be integrated as a single transgene or in concatamers, e.g., head-to-head tandems or head-to-tail tandems.

For a review of techniques that can be used to generate and assess transgenic animals, skilled artisans can consult Gordon (Intl. Rev. Cytol. 115:171-229, 1989) and may obtain additional guidance from, for example: Hogan et al. “Manipulating the Mouse Embryo” (Cold Spring Harbor Press, Cold Spring Harbor, N.Y., 1986; Krimpenfort et al, Bio/Technology 9: 86,1991; Palmiter et al., Cell 41: 343, 1985; Kraemer et al., “Genetic Manipulation of the Early Mammalian Embryo, “Cold Spring Harbor Press, Cold Spring Harbor, N.Y., 1985; Hammer et al., Nature 315: 680,1985; Purcel et al., Science, 244:1281, 1986; Wagner et al,. U.S. Pat. No. 5,175,385; and Krimpenfort et al,. U.S. Pat. No. 5,175,384.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gene Silencing Through Expression of Long Interrupted RNAS (“Bubble Hybrids”)

Given that RNAi-mediated gene silencing in C. elegans and Drosophila involves Dicer mediated cleavage of long dsRNAs to produce siRNAs the present inventors determine that transgenic long interrupted dsRNAs (bubble hybrids) are similarly processed to produce siRNAs capable of mediating gene inhibition, particularly without activation of the interferon response.

In exemplary embodiments, transgenic mice are generated that express unique complementary RNAs that can hybridize to form interrupted dsRNAs, (“bubble hybrids”). The transgenic mice are assayed for transgene expression, bubble hybrid formation and/or processing, and/or for coat color changes or cataracts/microphthalmia.

The bubble hybrid strategy is initially tested for RNAi of Rb in the lens. Lens-specific expression of Rb RNAi is accomplished by cloning the RNAi cassettes in the vicinity of a promoter, such as downstream of the alphaA-crystallin promoter (Reneker et al., 2000). If no phenotype is observed, there is the possibility that Rb synthesized before the activation of the alphaA-crystallin promoter persists at sufficient levels to obviate the effects of degradation of the mRNA. In this embodiment, the Rb RNAi transgene is placed under the control of another promoters, such as the Pax6 promoter, because its earlier activation may be effective at preventing the accumulation of Rb.

DNA constructs are generated that contain sequences from three different regions of the Rb (or tyrosinase) genes (see table below).

| | 5′ region | | | Central region | | | 3′ region | | |
|---|---|---|---|---|---|---|---|---|---|
| 30- | 61 | gaaaatgttcttggctgttttg | 82 | 837 | aaaatcctaacttactcagcc | 857 | 1599 | aaactagcataaaacatagacc | 1620 |
| 40% | | (SEQ ID NO:3) | | | (SEQ ID NO:4) | | | (SEQ ID NO:5) | |
| GC | | | | | | | | | |
| 50% | 155 | ttggcaaaagaatgctgcccac | 176 | 840 | atcctaacttactcagcccagc | 861 | 1535 | ggaaacagagtggactgaaagg | 1556 |
| GC | | (SEQ ID NO:6) | | | (SEQ ID NO:7) | | | (SEQ ID NO:8) | |
| 65- | 184 | gggtgatgggagtccctgcggc | 205 | 982 | agccaaaacccccaggctccc | 1002 | 1452 | ccccaggaggaaaggcagccac | 1473 |
| 75% | | (SEQ ID NO:9) | | | (SEQ ID NO:10) | | | (SEQ ID NO:11) | |
| GC | | | | | | | | | |

In specific embodiments, the length of the double stranded regions are shorter than about 30 basepairs, and the bubbles are flanked with UU-dinucleotides (Zamore et al., 2000) to favor cleavage by Dicer.

### Example 2

### Generation of Bubble Hybrids

The creation of bubble hybrids requires two pseudo-complementary transcripts. Two exemplary strategies are utilized to generate bubble hybrids that provide siRNA directed toward more than one site in the target mRNA.

Strategy 1—Double transgene bubble hybrid:

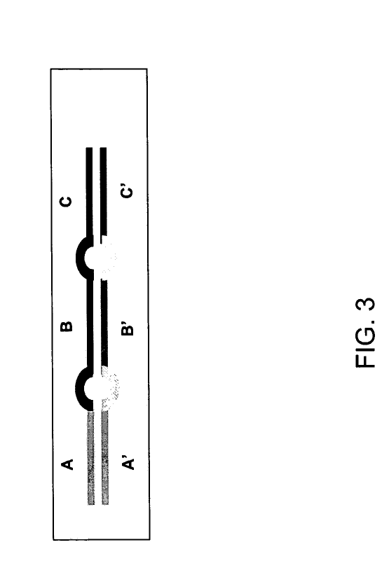

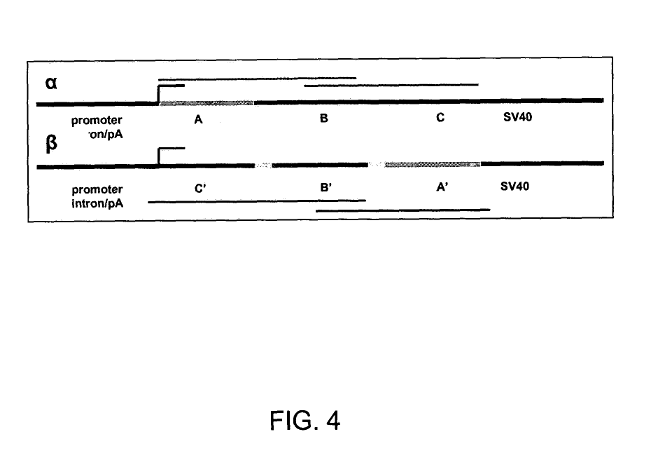

The first exemplary strategy is based on the generation of two transgenes, α and γ, that can hybridize as shown in FIG. 3. The a version contains “sense” sequences linked to the lens-specific (or melanocyte-specific) promoter. The γ, version has the corresponding “antisense” sequences, in reciprocal positions, linked to the same promoter. The two versions may differ in the intervening “bubble” sequences. The two vectors may be generated as described in FIG. 4 and co-injected to generate transgenic mice.

Strategy 2—Single transgene bubble hybrid:

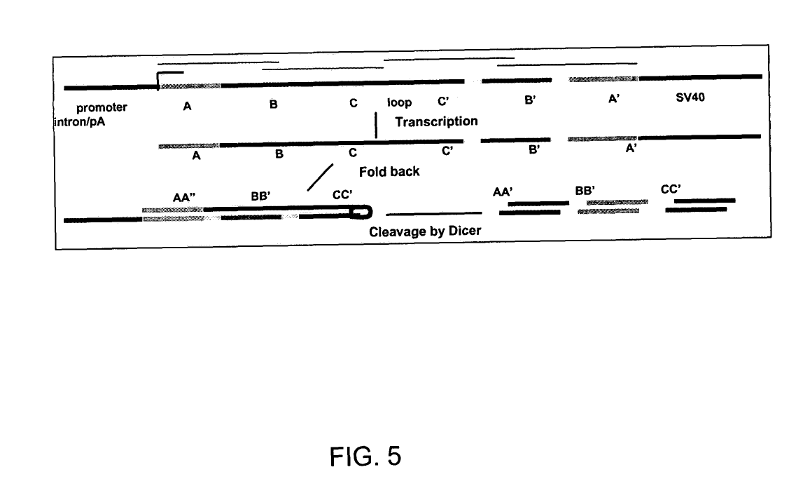

A second exemplary approach tests the efficacy of a single transgene that yields a transcript that can fold back onto itself to generate a long dsRNA. This design is analogous to the hairpin (or snap-back) dsRNAs that have been successfully used for heritable RNAi in Drosophila (Kennerdell et al., 2001) and C. elegans (Tavernarakis et al., 2000), with the crucial exception that instead of a long continuous dsRNA, the transgenes encode individual siRNAs separated by unpaired “bubbles” (FIG. 5).

In a specific embodiment, the bubble hybrid design is uniquely able to inactivate multiple genes simultaneously. Oligonucleotides A, B and C in FIGS. 3, 4 or 5 can derive from the same or different transcripts. This feature may be especially useful in situations where there is suspected or confirmed redundancy of function among several genes.

Thus, the bubble hybrid design is tested targeting expression of Rb in the lens in exemplary embodiments. The initial construct consists of three 50% GC 27-nucleotide segments, corresponding to three regions of the transcript. The effect of position is also tested by clustering the A, B and C sequences so that they are all targeted to the 5′ end, the middle or the 3′ end of the Rb transcript. A construct with A, B, and C sequences from introns of Rb is also tested. This produces a total of five exemplary double-transgene and single-transgene experiments.

### Example 3

### Assessment of Transgene Integration and Expression

Integration of the RNAi-encoding transgenes is determined by PCR on genomic DNA using primers SV40A and SV40B (Robinson and Overbeek, 1996) that amplify the SV40 sequences in the transgenes. These same primers flank the SV40 intron and are used for RT-PCR to test for transgene transcription. If the RT-PCR is positive, transgene expression is tested by in situ hybridization.

### Example 4

### Characterization of the Transgenic Mice

Transgenic mice are visually inspected for reduced fur and ocular pigmentation. If there is evidence that tyrosinase activity is reduced, the reduction in the level of tyrosinase expression is evaluated by Northern and Western blots (Sanbrook et al., 1989).

In specific embodiments, the mice are analyzed for specificity of gene silencing, transgene integration and expression, phenotype, integrity of the endogenous transcript, and interferon induction as described elsewhere herein. In addition, bubble hybrid mice are tested for the formation and processing of the bubble hybrid.

### Example 5

### Integrity of the Endogenous Transcript

The integrity of the tyrosinase or transcripts is evaluated by Northern blots with probes that will detect both the wildtype transcript (2.1 kb) and the transgenic RNAi transcript (approximately 1.1 kb). In specific embodiments, integration and expression of the RNAi transcript is concomitant with a reduction of target gene expression.

### Example 6

### Assay for Interferon Induction

Inhibition of gene expression may result from transgenic RNAi, but may also be a consequence of interferon induction by the dsRNA. Transgenic mice are analyzed for expression of interferon beta by RT-PCR or in situ hybridization. Expression analysis is facilitated by the massive increase in interferon-beta transcript resulting from the presence of positive feedback loops within the interferon pathway (Sen et al., 2001).

### Example 7

### Test for Formation of Bubble Hybrid and of siRNA

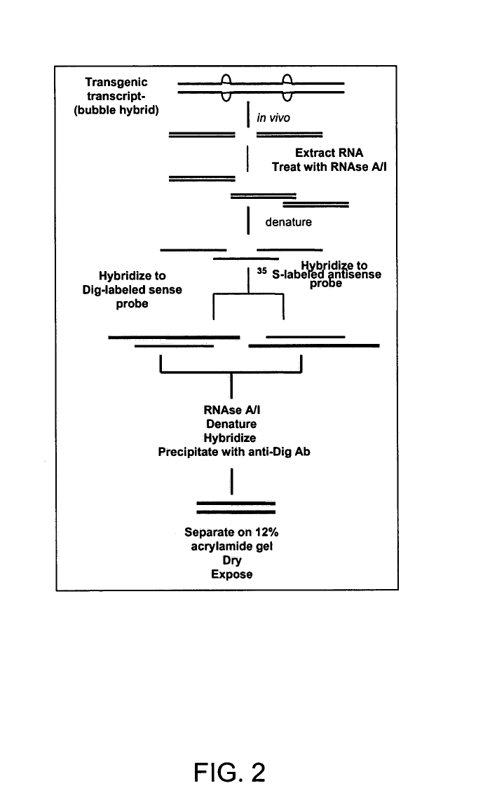

A compound RNAse protection assay is utilized to test for the presence of siRNAs (FIG. 2). In specific embodiments, the transgenic bubble hybrid is assembled and cleaved into about 21-22 nucleotide siRNAs. Total RNA is extracted and treated with RNAseA/I to degrade single stranded RNA. dsRNA is isolated using size fractionation columns, heat denatured and hybridized to two probes corresponding to the sense and antisense sequences of the targeted gene. One of these probes is labeled, such as with Digoxygenin, and the other one is also labeled, such as with ³⁵S. A second treatment with RNAse A/I degrades the non-hybridized probe fragments. Subsequent denaturation of the proband/probe duplexes, followed by re-annealing conditions generates double-label dsRNAs that can be immunoprecipitated with anti-Dig antibodies. Precipitated dsRNA is separated by electrophoresis, dried onto Whatmann paper and exposed for autoradiography. The detection of ³⁵S-labeled polynucleotides indicates the formation of the double labeled intermediate that is only possible if the cellular RNA extracts contain dsRNA species.

### Example 8

### Bubble Hybrid Design for RB RNAi

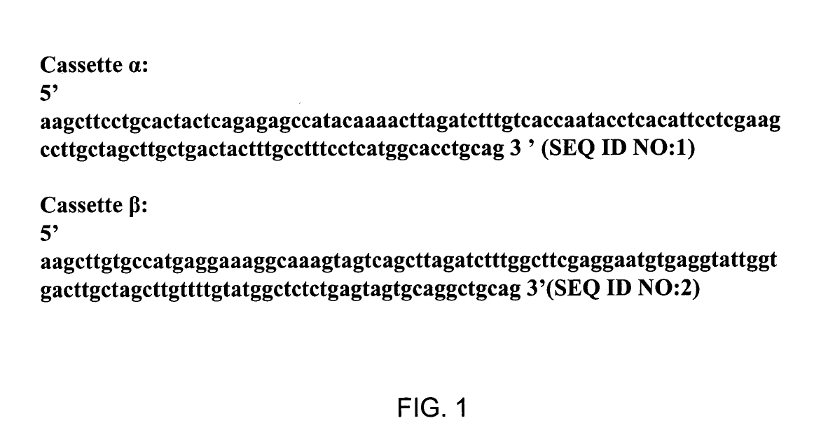

The present inventors designed bubble hybrid constructs to block Rb expression in the lens (see FIG. 1). The bubble hybrids are formed by hybridization of two different transcripts, in some embodiments. In exemplary embodiments, the transcripts have regions of homology (26-28 bp) separated by regions of non-homology (10 bp). In some embodiments, the interruption of the double stranded region by non-pairing sequences circumvents induction of an interferon response, and this bubble hybrid is processed to produce three siRNAs.

Given that it is known that multiple siRNAs with homology to various sequences of a target transcript are more effective than a single siRNA species, in an exemplary embodiment the Rb bubble hybrid transcripts contain sequences corresponding to the 5′ end, middle, and 3′ end of the Rb transcript. The non-pairing “bubbles” include two restriction sites not present elsewhere in the cassette or in the vector. These restriction sites are on alternate positions on the transcripts such that the corresponding sequences are not complementary. The sites are flanked by TT dinucleotides known to enhance the probability of cleavage by Dicer (Zamore et al., 2000).

### Example 9

### Additional Embodiments

The present invention in many embodiments comprises a bigenic system that can be used to obtain inducible, reversible, tissue-specific gene inactivation in the mouse. In some embodiments, transgenic mice in which the RNAi or stRNA cassettes for tyrosinase and Rb are under the control of TRE2 (tetracycline-inducible) or UAS (GAL4-inducible) promoters are produced and mated to transgenic mice expressing the tetracycline transactivator rTS2-2M (Urlinger et al., 2000) or the RU486-dependent GAL4 activation cassette (Tsai et al., 1998), under melanocyte- or lens-specific promoters. Double transgenic mice are treated with Doxycycline or RU486 and assayed for reversible RNAi-mediated inhibition of target gene expression.

In other embodiments, one impediment for the implementation of RNAi in the mouse is the existence of a mechanism guarding against dsRNA in mammalian cells: the interferon pathway. In specific embodiments, the activation of PKR and 2′, 5′-oligo(A) polymerase (Minks et al., 1979) is avoided with a transgene of the present invention. However, in some embodiments to inactivate PKR a dominant negative version of PKR, PKRΔE7 (Li et al., 2001), is introduced into the same target cell as the transgene. This creates cell-specific inhibition of the system for inactivating eiF2, thereby allowing the testing of the efficacy of longer dsRNAs for RNAi in the mouse. Alternative possibilities include the overexpression of known inhibitors of PKR such as p58, p67 or TRBP (Barber et al., 1994; Wu et al., 1996; Park et al., 1994) or the generation of RNAi transgenic mice on PKR (Yang et al., 1995) or interferon-beta (Deonarain et al., 2000) null background.

### REFERENCES

The following references, to the extent that they provide exemplary procedural or other details supplementary to those set forth herein, are specifically incorporated herein by reference.

### PATENTS

U.S. patent application Ser. No. US/2003/0084471

U.S. patent application Ser. No. US/2002/0162126

U.S. patent application Ser. No. US/2002/0086356

U.S. patent application Ser. No. US/2002/0173478

U.S. Pat. No. 6,573,099

WO 99/49029

WO 01/68836

WO 01/70949

WO 01/75164

WO 02/44321

WO 03/006477

WO 03/012052

### PUBLICATIONS

Ambros, V. (2001): Dicing up RNAs. Science 293: 811-813.

Barber, G N. (2001): Host defense, viruses and apoptosis. Cell Death and Differentiation 8: 113-126.

Bargmann, C. I. (2001): High-throughput reverse genetics:RNAi screens in Caenorhabditis elegans. Genome Biology 2: 1005.1-1005.3

Bass, B. L. (2001): The short answer. Nature 411: 428-429.

Baulcombe, D. (2001): Diced defence. Nature 409: 295-296.

Bernstein, E., Caudy, A. A., Hammond, S. M., and Hannon, G. J. (2001): Role for a bidentate ribonuclease in the initiation step of RNA interference. Nature 409: 363-366.

Bosher, J. M., and Labouesse, M. (2000): RNA interference: genetic wand and genetic watchdog. Nature Cell Biology 2: E31-E36

Caplen, N. J., Fleenor, J., Fire, A., and Morgan, R. A. (2000): dsRNA-mediated gene silencing in cultured Drosophila cells: a tissue culture model for the analysis of RNA interference. Gene 252: 95-105.

Caplen, N. J., Parrish, S., Imani, F., Fire, A., and Morgan, R. A. (2001): Specific inhibition of gene expression by small double-stranded RNAs in invertebrate and vertebrate systems. PNAS 98: 9742-9747.

Carthew, R. W. (2001): Gene silencing by double-stranded RNA. Current Opinion in Cell Biology 13: 244-248.

Der S D, Yang Y L, Weissmann C, Williams B R. (1997): A double-stranded RNA-activated protein kinase-dependent pathway mediating stress-induced apoptosis. Proc Natl Acad Sci U S A 94: 3279-3283.

Deonarain R, Alcami A, Alexiou M, Dallman M J, Gewert D R, Porter A C. (2000): Impaired antiviral response and alpha/beta interferon induction in mice lacking beta interferon. J Virol 2000 Apr;74(7):3404-3409.

Egwuagu C E, Sztein J, Chan C C, Mahdi R, Nussenblatt R B, Chepelinsky A B. (1994): gamma Interferon expression disrupts lens and retinal differentiation in transgenic mice. Dev Biol 166:557-568.

Elbashir, S. M., Harborth, J., Lendeckel, W., Yalcin, A., Weber, K., and Tuschl, T. (2001): Duplexes of 21-nucleotide RNAs mediate RNA interference in cultured mammalian cells. Nature 411: 494-498.

Elbashir, S. M., Lendeckel, W., and Tuschl, T. (2001): RNA interference is mediated by 21 and 22-nucleotide RNAs. Genes and Development 15: 188-200.

Fire, A., Xu, S., Montgomery, M. K., Kostas, S. A., Driver, S. E., and Mello, C. C. (1998): Potent and specific genetic interference by double-stranded RNA in Caenorhabditis elegans. Nature 391:806-811.

Franch, T., Petersen, M., Wagner, E. G., Jacobsen, J. P., and Gerdes, K. (1999): Antisense RNA regulation in prokaryotes:rapid RNA/RNA interaction facilitated by a general U-turn loop structure. Journal Molecular Biology 294: 1115-1125.

Fraser, A. G., Kamath, R. S., Zipperlen, P., Martinez-Campos, M., Sohrmann, M., and Ahringer, J. (2000): Functional genomic analysis of C. elegans chromosome I by systematic RNA interference. Nature 408: 325-330.

Fromm, L., Shawlot, W. Gunning, K. Butel, J. S. and Overbeek, P. A. (1994): The retinoblastoma binding region of simian virus 40 largeT antigen alters cell cycle regulation in lenses of transgenic mice. Mol. Cell Biol. 14:6743-6754.

Geiss, G., Jin, G., Guo, J., Bumgarner, R., Katze, M. G., and Sen, G. C. (2001): A comprehensive view of regulation of gene expresion by double-stranded RNA-mediated cell signaling. Journal of Biological Chemistry 276: 30178-30182.

Gil, J., and Esteban, M. (2000): Induction of apoptosis by the dsRNA-dependent protein kinase (PKR):mechanism of action. Apoptosis 5: 107-117.

Gonczy, P., Echeverri, G., Oegema, K., Coulson, A., Jones, S., Copley, R., Duperon, J., Oegema, J., Brehm, M., and Cassin, E. (2001): Functional genomic analysis of cell division in C. elegans using RNAi of genes on chromosome III. Nature 408: 331-336.

Grishok, A., Pasquinelli, A. E., Conte, D., Li, N., Parrish, S., Ha, I., Baillie, D. L., Fire, A., Ruvkun, G., and Mello, C. (2001): Genes and Mechanisms Related to RNA Interference Regulate Expression of the Small Temporal RNAs that Control C. elegans Developmental Timing. Cell 106: 23-34.

Grishok, A., Tabara, H., and Mello, C. C. (2000): Genetic Requirements for Inheritance of RNAi in C. elegans. Science 287: 2494-2497.

Gutell, R. R., Cannone, J. J., Konings, D., and Gautheret, D. (2000): Predicting U-turns in Ribosomal RNA with Comparative Sequence Analysis. Journal Molecular Biology 300: 791-803.

Hamada, M., Ohtsuka, T., Kawaida, R. et al. (2002): Effects on RNA Interference in Gene Expression (RNAi) in Cultured Mammalian Cells of Mismatches and the Introduction of Chemical Modifications at the 3′-Ends of siRNAs. Antisense and Nucleic Acid Drug Development 12:301-309.

Hammond, S. M., Bernstein, E., Beach, D., and Hannon, G. J. (2000): An RNA-directed nuclease mediates post-transcriptional gene silencing in Drosophila cells. Nature 404: 293-296.

Hammond, S. M., Boettcher, S., Caudy, A. A., Kobayashi, R., and Hannon, G. J. (2001): Argonaute2, a Link Between Genetic and Biochemical Analyses of RNAi. Science 293: 1146-1150.

Hammond, S. M., Caudy, A. A., and Hannon, G. J. (2001): Post-Transcriptional Gene Silencing By Double-Stranded RNA. Nature 2: 110-119.

Hasuwa, H., Kaseda, K., Einarsdottir, T., Okabe, M. (2002): Small Interfering RNA and Gene Silencing in Transgenic Mice and Rats. FEBS Letters 532:227-230.

Hope, I. A. (2001): Broadcast interference-functional genomics. Trends in Genetics 17: 297-299.

Hope, I. A. (2001): RNAi surges on:application to cultured mammalian cells. Trends in Genetics 17: 440-440.

Hunter T., Hunt, T., Jackson R J., and Roberston, H D. (1975): The characteristics of inhibition of protein synthesis by double-stranded ribonucleic acid in reticulocyte lysates. Journal of Biological Chemistry 250: 409-417.

Hunter, C. P. (1999): Genetics:A touch of elegance with RNAi. Current Biology 9: R440-R442.

Hutvagner, G., McLachlan, J., Pasquinelli, A. E., Balint, E., Tuschl, T., and Zamore, P. D. (2001): A Cellular Function for the RNA-Interference Enzyme Dicer in the Maturation of the let-7 Small Temporal RNA. Science 293: 834-838.

Kennerdell J R, Carthew R W. (1998): Use of dsRNA-mediated genetic interference to demonstrate that frizzled and frizzled 2 act in the wingless pathway. Cell 95:1017-1026.

Kennerdell J R, Carthew R W. (2000): Heritable gene silencing in Drosophila using double-stranded RNA. Nat Biotechnol 18:896-898.

Ketting, R. F., and Plasterk, R. H. A. (2000): A genetic link between co-suppression and RNA interference in C. elegans. Nature 404: 296-298.

Ketting R F, Fischer S E, Bernstein E, Sijen T, Hannon G J, Plasterk R H. (2001): Dicer functions in RNA interference and in synthesis of small RNA involved in developmental timing in C. elegans. Genes Dev 15(20):2654-2659.

Knight, S. W., and Bass, B. L. (2001): A Role for the RNase III Enzyme DCR-1 in RNA Interference and Germ Line Development in C. elegans. Science-express 1-3.

Kohara, M. I., Yamamoto, M., and Sugimota, A. (2001): Large-scale analysis of gene function in Caenorhabditis elegans by high-throughput RNAi. Current Biology 11:171-176.

Kuwabara, P. E., and Coulson, A. (2000): RNAi-Prospects for a General Technique for Determining Gene Function. Parasitology Today 16: 347-349.

Lagos-Quintana M, Rauhut R, Lendeckel W, Tuschl T (2001): Identification of Novel Genes Coding for Small Expressed RNAs. Science 294:853-858.

Lam G, Thummel C S. (2000): Inducible expression of double-stranded RNA directs specific genetic interference in Drosophila. Curr Biol 10:957-963.

Lau N C, Lim leEP, Weinstein E G, Bartel daVP. (2001): An Abundant Class of Tiny RNAs with Probable Regulatory Roles in Caenorhabditis elegans. Science 294:858-862.

Lee R C, Ambros V (2001): An Extensive Class of Small RNAs in Caenorhabditis elegans. Science 294:862-864.

Lee, E.-C., Yu, D., Martinez de Velasco, J., Tessarollo, L., Swing, D. A., Court, D. L., Jenkins, N. A., and Copeland, N. G. (2001): A Highly Efficient Escherichia coli-Based Chromosome Engineering System Adapted for Recombinogenic Targeting and Subcloning of BAC DNA. Genomics 73: 56-65.

Leirdal, M. and Sioud, M. (2002): Gene Silencing in Mammalian Cells by Preformed Small RNA Duplexes. Biochem. Biophys. Res. Commun. 295:744-748.

Li S, Koromilas A E. (2001): Dominant negative function by an alternatively spliced form of the interferon-inducible protein kinase PKR. J Biol Chem 276(17):13881-13890.

Li W, Nagineni C N, Efiok B, Chepelinsky A B, Egwuagu CE. (1999): Interferon regulatory transcription factors are constitutively expressed and spatially regulated in the mouse lens. Dev Biol 210:44-55.

Liegeois, N., Homer, J. W., and DePinho, R. A. (1996): Lens complementation system for the genetic analysis of growth, differentiation, and apoptosis in vivo. Genetics 93: 1303-1307.

Lin, R., and Avery, L. (1999): Policing rogue genes. Nature 402: 128-129.

Manche, L., Green, S. R., Schmedt, C., and Mathews, M. B. (1992): Interactions between double-stranded RNA regulators and the protein kinase DAI. Molecular Cellular Biology 12: 5238-5248.

Matsuda S, Ichigotani Y, Okuda T, Irimura T, Nakatsugawa S, Hamaguchi M. (2000): Molecular cloning and characterization of a novel human gene (HERNA) which encodes a putative RNA-helicase. Biochim Biophys Acta 2000 1490:163-169.

McCaffrey, J., Yamasaki, L., Dyson, N. J., Harlow, E., and Griep, A. E. (1999): Disruption of Retinoblastoma Protein Family Function by Human Papillomavirus Type 16 E7 Oncoprotein Inhibits Lens Development in Part through E2F-1. Molecular and Cellular Biology 19: 6458-6468.

Minks, M. A., West, D. K., Benvin, S., and Baglioni, C. (1979): Structural requirements of double-stranded RNA for the activation of 2′,5′-oligo(A) polymerase and protein kinase of interferon-treated HeLA cells. Journal of Biological Chemistry 254: 10180-10183.

Morel, J., and Vaucheret, H. (2000): Post-transcriptional gene silencing mutants. Plant Molecular Biology 43: 287-284.

Morgenbesser, S. D., Williams, B. O., Jacks, T., and DePinho, R. A. (1994): p53-dependent apoptosis produced by RB-deficiency in the developing mouse lens. Nature 37: 21-22.

Moss, E. G. (2001): RNA interference: It's a small RNA world. Current Biology 11: R772-R775.

Muyrers, J. P. P., Zhang, Y., Testa, G., and Stewart, A. F. (1999): Rapid modification of bacterial artificial chromosomes by ET-recombination. Nucleic Acids Research 27: 1555-1557.

Parrish, S. F. J., Xu, S., Mello, C., and Fire, A. (2000): Functional Anatomy of a dsRNA Trigger:Differential Requirement for the Two Trigger Strands in RNA Interference. Molecular Cell 6: 1077-1087.

Pasquinelli, A. E., Reinhart, B. J., Slack, F., Martindale, M. Q., Kuroda, M. I., Mailer, B., Hayward, D. C., Ball, E. E., Degnan, B., Muller, P., Spring, J., Srinivasan, A., Fishman, M., Finnerty, J., Corbo, J., Levine, M., Leahy, P., Davidson, E., and Ruvkun, G. (2000): Conversation of the sequence and temporal expression of let-7 heterochronic regulatory RNA. Nature 408: 86-89.

Patterson, M. (2001): Functional Genomics:Comprehensive interference. Nature Review Genetics 2: 6-6.

Paul, C. R., Good, P. D., Winer, I., Engelke, D. R. (2002): Effective Expression of Small Interfering RNA in Human Cells. Nat. Biotech. 29:505-508.

Piano, F., Schetter, A., Mangone, M., Stein, L., and Kemphues, K. (2000): RNAi analysis of genes expressed in the ovary of Caenorhabditis elegans. Current Biology 10: 1619-1622.

Plasterk, R. H. A., and Ketting, R. F. (2000): The silence of the genes. Current Opinion in Genetics and Development 10: 562-567.

Porter, S. D., and Meyer, C. J. (1994): A distal tyrosinase upstream element stimulates gene expression in neuralcrest-derived melanocytes of transgenic mice: position-independent and mosiac expression. Development 120: 2103-2111.

Porter, S. D., Hu, J., and Gilks, C. B. (1999): Distal Upstream Tyrosinase S/MAR-Containing Sequence Has Regulatory Properties Specific to Subsets of Melanocytes. Developmental Genetics 25: 40-48.

Reneker, L. W., Silversides, D. W., Xu, L., and Overbeek, P. A. (2000): Formation of corneal endothelium is essential for anterior segment development—a transgenic mouse model of anterior segment dysgenesis. Development 127: 533-542.

Ro, Y.-T., and Patterson, J. L. (2000): Identification of the Minimal Essential RNA Sequences Responsible for Site-Specific Targeting of teh Leishmania RNA Virus 1-4 Capsid Endoribonuclease. Journal of Virology 74: 130-138.

Robinson M L, Overbeek P A (1996): Differential expression of alpha A- and alpha B-crystallin during murine ocular development. Invest Ophthalmol Vis Sci 37: 2276-2284.

Rougvie, A. E. (2001): Control of Developmental Timing in Animals. Nature 2: 690-701.

Samuel, C. (2001): Antiviral actions of interferons. Clinical Microbiology Review 14: 778-809.

Sambrook J., Fritsch, E. F. and Maniatis, T. (1989): Molecular Cloning. Cold Spring Harbor Laboratory Press. Plainview, N.Y.

Schepers, U., and Kolter, T. (2001): RNA Interference: A New Way to Analyze Protein Function. Angewandte Chemie International Edition 40: 2437-2439.

Sen GC (2001): Viruses and interferons. Annu Rev Microbiol 55:255-281.

Sharp, P. A. (2001): RNA interference - - - 2001. Genes and Development 15: 485-490.

Sharp, P. A., and Zamore, P. D. (2000): RNA Interference. Science 287: 2432-2433.

Stewart, S. A., Dykxhoorn, D. M., Palliser, D., Mizuno, H., Yu, E. Y., An., D. S., Sabatini, D. M., Chen, I. S. Y., Hahn, W. C., Sharp, P. A., Weinberg, R. A., and Novina, C. D. (2003): Lentivirus-delivered Stable Gene Silencing by RNAi in Primary Cells. RNA 9:493-501.

Svoboda, P., Stein, P., Hayashi, H., and Schultz, R. M. (2001): Selective reduction of dormant maternal mRNAs in mouse oocytes by RNA interference. Development 127: 4147-4156.

Svoboda P, Stein P, Schultz R M.(2001): RNAi in Mouse Oocytes and Preimplantation Embryos: Effectiveness of Hairpin dsRNA. Biochem Biophys Res Commun 287(5):1099-1104.

Taniguchi T, Takaoka A (2001): A weak signal for strong responses: interferon-alpha/beta revisited. Nat Rev Mol Cell Biol 2:378-386.

Tavernarakis, N. W. S. L., Dorovkov, M., Ryazanov, A., and Driscoll, M. (2000): Heritable and inducible genetic interference by double-stranded RNA encoded by transgenes. Nature Genetics 24: 180-183.

Timmons, L., Court, D. L., and Fire, A. (2001): Ingestion of bacterially expressed dsRNAs can produce specific and potent genetic interference in Caenorhabditis elegans. Gene 103-112.

Tsai SY, O'Malley B W, DeMayo F J, Wang Y, Chua S S. (1998): A novel RU486 inducible system for the activation and repression of genes. Adv Drug Deliv Rev 30:23-31.

Tuschl, T., Zamore, P. D., Lehmann, R., Bartel, D. P., and Sharp, P. A. (1999): Targeted mRNA degradation by double-stranded RNA in vitro. Genes and Development 13: 3191-3197.

Ui-Tei K, Zenno S, Miyata Y, Saigo K (2000): Sensitive assay of RNA interference in Drosophila and Chinese hamster cultured cells using firefly luciferase gene as target. FEBS Lett 479:79-82.

Urlinger S, Baron U, Thellmann M, Hasan M T, Bujard H, Hillen W (2000): Exploring the sequence space for tetracycline-dependent transcriptional activators: novel mutations yield expanded range and sensitivity. Proc Natl Acad Sci U S A 97:7963-7968.

Wianny, F., and Zernicka-Goetz, M. (2000): Specific interference with gene function by double-stranded RNA in early mouse development. Nature Cell Biology 2: 70-75.

Williams, B. R. (1999): PKR;a sentinel kinase for cellular stress. Oncogene 18: 6112-6120.

Wilson, I W, Siemering, K R, Praszkier, J and Pittard, A J (1997): Importance of Structural Differences between Complementary RNA Molecules to Control Replication of an IncB Plasmid. J. Bacteriol. 179(3):742-753.

Yang Y L, Reis L F, Pavlovic J, Aguzzi A, Schafer R, Kumar A, Williams B R, Aguet M, Weissmann C. (1995): Deficient signaling in mice devoid of double-stranded RNA-dependent protein kinase. EMBO J 14(24):6095-6106.

Yang, X. W., Model, P., and Heintz, N. (1997): Homologous recombination based modification in Esherichia coli and germline transmission in transgenic mice of a bacterial artificial chromsome. Nature Biotechnology 15: 859-865.

Yang S, Tutton S, Pierce E, Yoon K. (2001): Specific double-stranded RNA interference in undifferentiated mouse embryonic stem cells. Mol Cell Biol 22:7807-7816.

Yokoyama, T., Silversides, D. W., Waymire, K. G., Kwon, B. S., Takeuchi, T., and Overbeek, P. A. (1990): Conserved cysteine to serine mutation in tyrosinase is responsible for the classical albino mutation in laboratory mice. Nucleic Acids Research 18: 7293-7298.

Zamore, P. D. (2001): RNA interference:listening to the sound of silence. Nature Structural Biology 8: 746-750.

Zamore, P. D., Tuschl, T., Sharp, P. A., and Bartel, D. P. (2000): RNAi: Double-Stranded RNA Directs the ATP-Dependent Cleavage of mRNA at 21 to 23 Nucleotide Intervals. Cell 101: 25-33.

Zhang, Y., Buchholz, F., Muyrers, J. P. P., and Stewart, A. F. (1998): A new logic for DNA engineering using recombination in Escherichia coli. Nature Genetics 20: 123-128.

Zhao, S., and Overbeek, P. A. (1999): Tyrosinase-Related Protein 2 Promoter Targets Transgene Expression to Ocular and Neural Crest-Derived Tissues. Development Biology 216: 154-163.

Zipperlen, P., Fraser, A. G., Kamath, R. S., Martinez-Campos, M., and Ahringer, J. (2001): Roles for 147 enbryonic lethal genes on C.elegans chromosome I identified by RNA interference and video microscopy. The EMBO Journal 20: 3984-3992.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An RNA composition that comprises at least one double strand region, wherein said double stranded region is interrupted by at least one region of non-complementarity, wherein said composition induces destruction of a target nucleic acid sequence.

2. The RNA composition of claim 1, wherein said target nucleic acid sequence is a transcript.

3. The RNA composition of claim 1, wherein said composition is substantially incapable of eliciting an interferon pathway in a cell.

4. The RNA composition of claim 1, wherein said composition comprises one RNA molecule.

5. The RNA composition of claim 1, wherein said composition comprises two or more RNA molecules.

6. The RNA composition of claim 1, wherein said composition is further defined as comprising a construct, said construct having in a 5′ to 3′ orientation:
a first double stranded RNA region;
a first region of non-complementarity;
a second double stranded RNA region;
a second region of non-complementarity; and
a third double stranded RNA region.

7. The composition of claim 6, wherein said RNA composition comprises at least one regulatory sequence operably linked to the construct.

8. The composition of claim 7, wherein the regulatory sequence is a constitutive promoter, an inducible promoter, a tissue-specific promoter, or a combination thereof.

9. The RNA composition of claim 1, wherein said one or more double stranded RNA regions are at least about 22 nucleotides in length.

10. The RNA composition of claim 1, wherein said one or more double stranded RNA regions are between about 22 and about 30 nucleotides in length.

11. The RNA composition of claim 1, wherein said one or more double stranded RNA regions are between about 27 and about 30 nucleotides in length.

12. The RNA composition of claim 1, wherein said one or more regions of non-complementarity are at least about 5 nucleotides in length.

13. The RNA composition of claim 1, wherein said one or more regions of non-complementarity are from about 5 to about 12 nucleotides in length.

14. The RNA composition of claim 1, wherein said one or more double stranded RNA regions are complementary to a target nucleic acid sequence.

15. The RNA composition of claim 14, wherein said one or more double stranded RNA regions are complementary to the same target nucleic acid sequence.

16. The RNA composition of claim 14, wherein said one or more double stranded RNA regions are complementary to different target nucleic acid sequences.

17. The RNA composition of claim 14, wherein said one or more double stranded RNA regions are fully complementary to a target nucleic acid sequence.

18. The RNA composition of claim 14, wherein said one or more double stranded RNA regions are complementary to a 5′ region of a target transcript.

19. The RNA composition of claim 18, wherein said 5′ region is a 5′ untranslated region of a target transcript.

20. The RNA composition of claim 14, wherein said one or more double stranded RNA regions are complementary to a 3′ region of a target transcript.

21. The RNA composition of claim 20, wherein said 340 region is a 3′ untranslated region of a target transcript.

22. The RNA composition of claim 1, wherein said composition is encoded by a single transgene.

23. The RNA composition of claim 5, wherein said composition is encoded by two transgenes.

24. The RNA composition of claim 1, wherein the junction between at least one double stranded RNA region and at least one region of non-complementarity comprises at least two consecutive T's.

25. The RNA composition of claim 1, wherein said composition is further defined as comprising n number of double stranded regions and n-1 number of regions of non-complementarity.

26. A vector comprising the RNA composition of claim 1.

27. A transgene comprising the RNA composition of claim 1.

28. A mammalian cell comprising the RNA composition of claim 1.

29. A transgenic, non-human animal having at least one cell comprising a transgene encoding a RNA composition of claim 1, wherein the transgene is expressed in one or more cells of the transgenic animal, resulting in inducing destruction of at least one target nucleic acid sequence by the RNA composition.

30. An RNA composition comprising two or more double strand regions, adjacent regions of which are separated from each other by one or more regions of non-complementarity, wherein at least two of said double strand regions are complementary to at least two different target transcripts, wherein said RNA composition is capable of inducing destruction of said transcripts.

31. The RNA composition of claim 30, wherein said double stranded regions are fully complementary to said transcripts.

32. A vector having a promoter that operably regulates sequence that encodes an RNA, wherein said sequence comprises one or more nucleic acid sequence constructs each of which are flanked by at least two restriction enzyme sites, wherein upon intramolecular hybridization of said RNA, at least one of said constructs generates a region of non-complementarity within said RNA.

33. The vector of claim 32, wherein said two restriction enzyme sites are non-identical.

34. The vector of claim 32, wherein said sequence comprises a signal for poly (A) addition.

35. The vector of claim 32, wherein said sequence is further defined as having the following components present in a 5′ to 3′ orientation:
a) a first restriction enzyme site;
b) a second restriction enzyme site;
c) sequence that encodes one strand of a first region of non-complementarity;
d) a third restriction enzyme site;
e) a fourth restriction enzyme site;
f) sequence that encodes one strand of a second region of non-complementarity;
g) a fifth restriction enzyme site;
h) a sixth restriction enzyme site;
i) sequence that encodes one strand of a third region of non-complementarity;
j) a loop region;
k) sequence that encodes a second strand of the third region of non-complementarity, wherein the sequence is non-complementary to the sequence in i);
l) a seventh restriction enzyme site;
m) an eighth restriction enzyme site;
n) sequence that encodes a second strand of the second region of non-complementarity, wherein the sequence is non-complementary to the sequence in f);
o) a ninth restriction enzyme site;
p) a tenth restriction enzyme site;
q) sequence that encodes a second strand of the first region of non-complementarity, wherein the sequence is non-complementary to the sequence in c); and
r) sequence that directs addition of a poly A tail.

36. A kit comprising the vector of claim 32.

37. The kit of claim 36, wherein said kit further comprises one or more restriction enzymes.

38. The kit of claim 37, wherein said kit further comprises a buffer suitable for at least one restriction enzyme.

39. A eukaryotic cell exhibiting a target nucleic acid sequence-specific knockout phenotype, wherein said cell is transfected with at least one RNA composition capable of and under conditions suitable for inducing destruction of the target nucleic acid sequence, wherein the RNA composition comprises at least one double stranded region interrupted by at least one region of non-complementarity.

40. The cell of claim 39, wherein said cell is in a eukaryotic non-human organism.

41. An isolated genetic construct that is capable of inducing destruction of at least one target nucleic acid sequence in an animal cell that is transfected with said construct, wherein the genetic construct comprises nucleic acid sequence comprising or encoding a RNA composition, said RNA composition comprising:
a first double strand region that is substantially identical to at least a region of a first target nucleic acid sequence; and
a second double strand region that is substantially identical to at least a region of a second target nucleic acid sequence, said first and second double stranded regions separated by a region of non-complementarity, and wherein the double strand regions are under the control of at least one operable promoter.

42. The construct of claim 41, wherein the first and second target nucleic acid sequences are transcripts from the same gene or locus.

43. The construct of claim 41, wherein the first and second target nucleic acid sequences are transcripts from a different gene or locus.

44. The construct of claim 41, wherein said first and second double stranded regions are under the control of different operable promoters.

45. A method of inducing destruction of a target nucleic acid sequence in an animal cell, comprising expressing in said animal cell a genetic construct of claim 41.

46. A method of inducing destruction of at least one target nucleic acid sequence in a cell, comprising introducing to the cell an effective amount of a RNA composition comprising one or more double stranded RNA regions each of which are substantially identical to a portion of a target nucleic acid sequence, and each of which said double stranded regions are separated by the adjacent double stranded RNA region by a region of non-complementarity, wherein upon said introducing said RNA composition to the cell, said composition induces destruction of said target nucleic acid sequence.

47. The method of claim 46, wherein the cell is in a mammal.

48. The method of claim 47, wherein the mammal is a mouse.

49. A method of preparing an RNA composition of claim 1, comprising the steps of:
synthesizing two RNA strands, wherein said RNA strands are capable of forming a double stranded RNA molecule; and
combining the synthesized RNA strands under conditions wherein a double stranded RNA molecule is produced, said double stranded RNA molecule capable of inducing destruction of a target nucleic acid sequence.

50. The method of claim 49, wherein said RNA strands are chemically synthesized.

51. The method of claim 49, wherein said RNA strands are enzymatically synthesized.

52. The method of claim 49, wherein said combining step occurs in a cell following introduction into the cell of the two RNA strands or nucleic acids encoding them.

53. A method of preparing a single stranded RNA composition of claim 1, comprising the steps of:
obtaining at least one region of a nucleic acid encoding said RNA composition;
obtaining at least another region of a nucleic acid encoding said RNA composition;
cloning said regions operably together in a vector to produce a single transgene, wherein said vector comprises at least one regulatory sequence operably linked to said transgene; and
expressing said RNA composition.

54. A method of mediating RNA interference of a nucleic acid sequence in a cell or organism, comprising:
introducing into the cell or organism at least one RNA composition, wherein the RNA composition comprises in a 5′ to 3′ direction at least:
a first double stranded region;
a region of non-complementarity; and
a second double stranded region, wherein at least one of the double stranded regions targets the nucleic acid sequence for degradation; and
maintaining the cell or organism under conditions wherein degradation of the target nucleic acid sequence occurs.

55. The method of claim 54, wherein said nucleic acid sequence encodes a cellular mRNA or a viral mRNA.

56. A method of inducing destruction of nucleotide sequence from more than one locus, comprising:
introducing into the cell or organism at least one RNA composition, wherein the RNA composition comprises in a 5′ to 3′ direction at least:
a first double stranded region;
a region of non-complementarity; and
a second double stranded region, wherein the double stranded regions target different nucleic acid sequences for degradation; and
maintaining the cell or organism under conditions wherein the nucleotide sequences are destroyed.

57. The method of claim 56, wherein said method is further defined as destroying a transcript from more than one gene.


SEQUENCE LISTING (12 sequences)

SEQ ID NO: 1 - DNA, 119 bp, Artificial Sequence
  Synthetic Artificial Construct
aagcttcctgcactactcagagagccatacaaaacttagatctttgtcaccaatacctcacattcctcgaagccttgctagcttgctgactactttgcctttcctcatggcacctgcag

SEQ ID NO: 2 - DNA, 119 bp, Artificial Sequence
  Synthetic Artificial Construct
aagcttgtgccatgaggaaaggcaaagtagtcagcttagatctttggcttcgaggaatgtgaggtattggtgacttgctagcttgttttgtatggctctctgagtagtgcaggctgcag

SEQ ID NO: 3 - DNA, 22 bp, Artificial Sequence
  Synthetic Artificial Construct
gaaaatgttcttggctgttttg

SEQ ID NO: 4 - DNA, 21 bp, Artificial Sequence
  Synthetic Artificial Construct
aaaatcctaacttactcagcc

SEQ ID NO: 5 - DNA, 22 bp, Artificial Sequence
  Synthetic Artificial Construct
aaactagcataaaacatagacc

SEQ ID NO: 6 - DNA, 22 bp, Artificial Sequence
  Synthetic Artificial Construct
ttggcaaaagaatgctgcccac

SEQ ID NO: 7 - DNA, 22 bp, Artificial Sequence
  Synthetic Artificial Construct
atcctaacttactcagcccagc

SEQ ID NO: 8 - DNA, 22 bp, Artificial Sequence
  Synthetic Artificial Construct
ggaaacagagtggactgaaagg

SEQ ID NO: 9 - DNA, 22 bp, Artificial Sequence
  Synthetic Artificial Construct
gggtgatgggagtccctgcggc

SEQ ID NO: 10 - DNA, 21 bp, Artificial Sequence
  Synthetic Artificial Construct
agccaaaacccccaggctccc

SEQ ID NO: 11 - DNA, 22 bp, Artificial Sequence
  Synthetic Artificial Construct
ccccaggaggaaaggcagccac

SEQ ID NO: 12 - DNA, 3308 bp, Mouse
caaagaagactgtgacactcattaacctattggtgcagattttgtatgatctaaaggagaaatgttcttggctgttttgtattgccttctgtggagtttccagatctctgatggccattttcctcgagcctgtgcctcctctaagaacttgttggcaaaagaatgctgcccaccatggatgggtgatgggagtccctgcggccagctttcaggcagaggttcctgccaggatatccttctgtccagtgcaccatctggacctcagttccccttcaaaggggtggatgaccgtgagtcctggccctctgtgttttataataggacctgccagtgctcaggcaacttcatgggtttcaactgcggaaactgtaagtttggatttgggggcccaaattgtacagagaagcgagtcttgattagaagaaacatttttgatttgagtgtctccgaaaagaataagttcttttcttacctcactttagcaaaacatactatcagctcagtctatgtcatccccacaggcacctatggccaaatgaacaatgggtcaacacccatgtttaatgatatcaacatctacgacctctttgtatggatgcattactatgtgtcaagggacacactgcttgggggctctgaaatatggagggacattgattttgcccatgaagcaccagggtttctgccttggcacagacttttcttgttattgtgggaacaagaaattcgagaactaactggggatgagaacttcactgttccatactgggattggagagatgcagaaaactgtgacatttgcacagatgagtacttgggaggtcgtcaccctgaaaatcctaacttactcagcccagcatccttcttctcctcctggcagatcatttgtagcagatcagaagagtataatagccatcaggttttatgcgatggaacacctgagggaccactattacgtaatcctggaaaccatgacaaagccaaaacccccaggctcccatcttcagcagatgtggaattttgtctgagtttgacccagtatgaatctggatcaatggatagaactgccaatttcagctttagaaacacactggaaggatttgccagtccactcacagggatagcagatccttctcaaagtagcatgcacaatgccttacatatctttatgaatggaacaatgtcccaagtacagggatcggccaacgatcccatttttcttcttcaccatgcttttgtggacagtatttttgaacaatggctgcgaaggcaccgccctcttttggaagtttacccagaagccaatgcacctatcggccataacagagactcttacatggttcctttcataccgctctatagaaatggtgatttcttcataacatccaaggatctgggatatgactacagctacctccaagagtcagatccaggcttttacagaaattatattgagccttacttggaacaagccagtcgtatctggccatggcttcttggggcagcactggtgggagctgttattgctgcagctctctctgggcttagcagtaggctatgccttcagaagaagaagaagaagaagcaaccccaggaggaaaggcagccactcctcatggacaaagacgactaccacagcttgctgtatcagagccatctgtgaacatcctaggaaacagagtgggactgaaaggttttacctcactcgacctatttgttggtgtttctacaaatttaaactagtataaaacatagaccatagctgtttggctttttttcagacccatgttttttcctaagtcctagtttctaagaaatgactgggatttgctaaaatatatatatatataaataataacttactaatagctaaataaaatttcctcttacaactaattgagctggtttttatgaatgtgtcttaattatttaaacttgaggcacatttttgttttccttacttcattgtgaatttccaagaaaaatattctctctctctctctctctcgtgtgtttgtgtgtatgtgtgtgttaactgattcaaacaattttgaaaatcttggattgatagaaatgattcattaatttatgaaattatttcattaatgattaggaaagacgaataattactaaattagtaacagaggagaacatctgccagcttttaattaaattgtcatttaagttaccttatctaccttctgtgactggtggaaaaatatcaggcaagagatgggaatgctctgcctaataggatagtggctcctggaaggagtgggttattactagagattattacctgaagtttaccatagttagaaaattaatcaaaacagatgactcagtaacatctgaagcttcaagtcggcttgactgcaatctgaaatcatcaagcccaagagccaaaggaatgggaacagcgatgggaaactatctgaatcagattctagtgtgatagtgtcaggggcacatgggtcatctttgagaccttcacacctgttgagtcaccaaaatttgctgtgaatgtaaatttttactgtaaattaattttttcttttctttttaaaaagatttatttattattatacataagtacactgtagctgtcttcagacacaccagaagagggtgtcagatctcattacagatggttgtgagccaccatgtggttgctgggatttgaactcaggacctctggaagaacagtcagtgctcttacccgctgagccatctcgccagtcccagtaaatttttactttagtgaaagtaaaatttaagttttagtttttagtttagtaaaattttaggaagcaaatttttagttttctaaactaatttttttttctagtactggacatcaacccagtgccttgtatatgcaatgcaagcattttcttgtactctgctacctagcatgtatatataaatctacccaacaaatgttcattacagctgacaagggtctttataaactcagtgtttccctttatcacaatacaattccctcctttgccacttcatgtcatcatagaatattgtttttttctctagcggttcaaggtatgtatttgtatagcagtcacacctttgataaaagttaccatctctttgattatatatctcattatggtaacaaaattatattatgactatttcaatatatctgaaagtttcattaaattctcattaactttgtatatttcagtcttgcttattgtgaagcttttataaattgcttcactttttttctgaaattgtcctgttgctacatcattctgttaagaaataaataagtggcaatattttcc